(19) United States
(12) Patent Application Publication
(10) Pub. No.: US 2020/0179541 A1
(43) Pub. Date: Jun. 11, 2020

(54) METHODS AND COMPOSITIONS FOR ENZYME-MEDIATED SITE-SPECIFIC RADIOLABELING OF GLYCOPROTEINS

(71) Applicant: Life Technologies Corporation, Carlsbad, CA (US)

(72) Inventors: Brian AGNEW, Eugene, OR (US); Robert AGGELER, Eugene, OR (US); Hee Chol KANG, Eugene, OR (US); Aimei CHEN, Eugene, OR (US); Kyle GEE, Springfield, OR (US)

(21) Appl. No.: 16/713,388

(22) Filed: Dec. 13, 2019

Related U.S. Application Data
(63) Continuation of application No. 15/826,593, filed on Nov. 29, 2017, now abandoned.
Application No. 15/826,593 is a continuation of application No. 15/665,885, filed on Aug. 1, 2017, now abandoned.
Application No. 15/665,885 is a continuation of application No. 14/425,114, filed on Mar. 2, 2015, now abandoned.
(60) Provisional application No. 61/718,576, filed on Oct. 25, 2012.

(51) Int. Cl. A61K 51/10; G01N 33/534; G01N 33/68; C07K 14/435; C07K 16/18; C12P 21/00; C12P 21/06; G01N 33/58

(52) U.S. Cl. CPC A61K 51/10; G01N 33/534; G01N 33/6854; G01N 33/582; C07K 16/18; C12P 21/005; C12P 21/06; C07K 14/435

(57) ABSTRACT

Provided herein are methods, compositions and kits for use in the site-specific labeling of glycoproteins comprising a combination of enzyme-mediated incorporation of modified sugars comprising a chemical handle and cycloaddition chemistry with a labeling molecule comprising a reactive group, a metal ion chelator, and/or a fluorophore.

### RELATED APPLICATIONS

This application is a Continuation of U.S. Non Provisional Application No. 15/826,593, filed on Nov. 29, 2017, which is a Continuation of U.S. Non Provisional Application No. 15/665,885, filed on Aug. 1, 2017, which is a Continuation of U.S. Non Provisional Application No. 14/425,114, filed Mar. 2, 2015, which is a U.S. National Stage Application of PCT Application Serial No. PCT/US2013/066765, filed Oct. 25, 2013, which claims the benefit of U.S. Provisional Application No. 61/718,576, filed Oct. 25, 2012, the disclosures of which are incorporated herein by reference.

### FIELD

This invention relates to the field of site-specific radiolabeling of glycoproteins.

### BACKGROUND

The remarkable specificity, affinity and stability of antibodies have made them extremely promising vectors for the delivery of diagnostic and therapeutic radioisotopes to tumors. Indeed, over the past two decades, antibodies bearing radionuclides ranging from ¹²⁴I, ¹¹¹In, and ⁶⁴Cu for PET and SPECT imaging and ⁹⁰Y, ¹⁷⁷Lu, and ²²⁵Ac for radiotherapy have been translated to the clinic.

One potential limitation to the development and translation of radioimmunoconjugates, however, is the lack of site-specificity in the radiolabeling of antibodies. The vast majority of antibody radiolabeling methods rely on reactions with amino acid residues, typically either tyrosines for radioiodinations or lysines or cysteines for radiometal chelator conjugations. Yet antibodies are, of course, very large molecules and thus possess multiple copies of each of these amino acids. Consequently, precise control over the specific molecular location of the radionuclides or radiometal-chelator complexes on the antibody is impossible.

This lack of radiolabeling site-specificity presents two principal complications to the development and use of radioimmunoconjugates. First, without the ability to control the precise location of the radiolabeling on the antibody, radioisotopes or radiometal chelates may end up appended to the antigen-binding region of the antibody, adversely affecting the immunoreactivity of the construct. Second, without knowledge of the exact site of radiolabeling, the resultant radioimmunoconjugates remain somewhat inadequately chemically defined, which can become a problem both in basic scientific investigations and, perhaps more importantly, during the clinical regulatory review and approval process.

Furthermore, the lack of reproducibility offered by either direct labeling reactions or chelator conjugation strategies presents another troubling limitation to current strategies for the construction of radioimmunoconjugates. Given the non-site-selective nature of most radiolabeling methodologies, each new immunoconjugate must undergo extensive optimization in order to obtain a degree of labeling that strikes a suitable balance between the specific activity of the final radioimmunoconjugate and its immunoreactivity, a process which can be time-consuming, tedious, and costly. Furthermore, the reactive chemical conjugation reagents, themselves, tend to be chemically unstable and subject to hydrolysis, thus requiring storage under inert atmospheres of argon or nitrogen, and can result in compounding poor reproducibility issues upon repeated usage.

Optical imaging can be a valuable complement to PET, especially as a visual aid for tumor resection. Imaging in the near-infrared (NIR, 700-900 nm) region is optimal for in vivo applications as the absorbance spectra for all biomolecules reach a minimum providing a clear optical window for small animal studies and various human clinical scenarios (e.g., breast imaging, endoscopy, surgical guidance, etc.). In addition to better tissue penetration of light, there is also significantly less tissue autofluorescence in the NIR window. One valuable clinical application where dual-labeled imaging is particularly useful is through the primary use of whole-body PET imaging to identify the location of tumor(s) and subsequent NIR fluorescence (NIRF) imaging to guide tumor resection. Additionally, the use of fluorescent dyes across the full spectrum of wavelengths can be used for imaging tumors on the surface of tissues for instance in the gastrointestinal system, on the skin, or in the eye.

Standard conjugation with optical imaging probes suffers the same shortcomings of non-site-selectivity and poor reproducibility, especially from antibody-to-antibody. And furthermore, dual conjugation approaches utilizing both an optical imaging probe and a radiolabeling probe involve parallel or serial rounds of antibody conjugation reactions, compounding these issues. For instance, determining the correct balance of chelate conjugation with a NIR-dye would require multiple rounds of optimization to determine the optimal degree of labeling for each of the detection probes while at the same time maintaining antibody binding affinity, a process that would need to be repeated for each new immunoconjugate. As such, when using standard conjugation techniques the optimal degree of labeling cannot always be reached due to the requirement to maintain antigen binding affinity.

In response to these problems, efforts have been made to develop site-specific radiolabeling methodologies for antibodies. With few exceptions, these strategies rely on antibodies that have been specially engineered to bear either reactive thiol moieties or fusion proteins; however, the use of genetically engineered antibodies adds undue layers of complexity and expense that hamper both the modularity and potential for clinical translation of the systems. Thus, a need exists for robust and reliable methods for site-selective radiolabeling of glycoproteins and antibodies that do not require special antibody engineering.

### SUMMARY

Herein are provided methods, compositions and kits for use in the site-specific labeling of glycoproteins comprising a combination of enzyme-mediated incorporation of modified sugars comprising a chemical handle and cycloaddition chemistry with a labeling molecule comprising: a metal ion chelator group and a reactive group that attaches to the chemical handle of the modified sugar; a fluorophore and a reactive group that attaches to the chemical handle of the modified sugar; or a metal ion chelator, a reactive group that attaches to the chemical handle of the modified sugar, and a fluorophore. In certain embodiments, the glycoprotein comprises a terminal GlcNAc residue. In certain embodiments, the glycoprotein is an antibody or an Fc-fusion protein. In certain embodiments, the antibody is an IgA, an IgE, an IgD, an IgG, an IgM, or an IgY. In certain embodiments, the antibody has an affinity for a cell-associated antigen. In certain embodiments, the terminal GlcNAc residues are present on the Fc region of the antibody.

In certain embodiments, methods for labeling a glycoprotein are provided, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group and a reactive group;

e) contacting the modified glycoprotein with the labeling molecule, wherein the reactive group attaches to the chemical handle to provide a labeled glycoprotein;

f) providing a radioactive metal ion; and

g) contacting the labeled glycoprotein with the radioactive metal ion, wherein the metal ion associates with the chelator group to provide a radiolabeled glycoprotein.

In certain embodiments, the labeling molecule further comprises a fluorophore. In certain embodiments, the glycoprotein comprises an antibody or an Fc-fusion protein. In certain embodiments, the antibody is an IgA, an IgE, an IgD, an IgG, an IgM, or an IgY. In certain embodiments, the antibody has an affinity for a cell-associated antigen.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, the modified sugar is attached to the terminal GlcNAc residue by a galactosyl transferase. In certain embodiments, the galactosyl transferase is a mutant galactosyl transferase. In certain embodiments, the galactosyl transferase is a Y289L mutant galactosyl transferase.

In certain embodiments, methods for labeling a glycoprotein are provided, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group, a reactive group, and a fluorophore;

e) contacting the modified glycoprotein with the labeling molecule, wherein the reactive group attaches to the chemical handle to provide a labeled glycoprotein;

f) providing a radioactive metal ion; and

g) contacting the labeled glycoprotein with the radioactive metal ion, wherein the metal ion associates with the chelator group to provide a radiolabeled glycoprotein.

In certain embodiments, the glycoprotein comprises an antibody or an Fc-fusion protein. In certain embodiments, the antibody is an IgA, an IgE, an IgD, an IgG, an IgM, or an IgY. In certain embodiments, the antibody has an affinity for a cell-associated antigen.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, the chemical handle comprises an azide group, and the reactive group comprises a terminal triarylphosphine, an alkyne, a terminal alkyne, or an activated alkyne group. In certain embodiments, the chemical handle comprises a terminal triarylphosphine, an alkyne, a terminal alkyne or an activated alkyne group, and the reactive group comprises an azide group. In certain embodiments, the activated alkyne comprises a cyclooctyne group, a difluorocyclooctyne group, a dibenzocyclooctyne group, an aza-dibenzocyclooctyne group, or a cyclononyne group. In certain embodiments, the activated alkyne group comprises a dibenzocyclooctyne group. In certain embodiments, the dibenzocyclooctyne group is 4-dibenzocyclooctynol (DIBO). In certain embodiments, the chemical handle comprises a Diels-Alder diene and the reactive group comprises a Diels-Alder dienophile. In certain embodiments, the chemical handle comprises a Diels-Alder dienophile and the reactive group comprises a Diels-Alder diene. In certain embodiments, the chemical handle comprises a straight chain or branched chain C₁-C₁₂ carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR₂(C═O)NR₃NH₂ (carbazide), —NR¹NR²(C═S)NR³NH₂ (thiocarbazide), or —ONH₂ (aminooxy), wherein each R¹, R² and R³ is independently H or alkyl having 1-6 carbons. In certain embodiments, the modified sugar comprising a chemical handle is UDP-GalNAz. In certain embodiments, the modified sugar comprising a chemical handle is UDP-GalKyne. In certain embodiments, the modified sugar comprising a chemical handle is UDP-GalKetone.

In certain embodiments, the metal chelating group is selected from the group consisting of a metal chelating dimer, a metal chelating trimer, a metal chelating oligomer, and a metal chelating polymer. In certain embodiments, the metal ion chelator group comprises a group selected from the group consisting of 1,4,8,11-tetraazabicyclo[6.6.2]hexadecane-4,11-diyl)diacetic acid (CB-TE2A); desferrioxamine (DFO); diethylenetriaminepentaacetic acid (DTPA); 1,4,7,10-tetraazacyclotetradecane-1,4,7,10-tetraacetic acid (DOTA); ethylenediaminetetraacetic acid (EDTA); ethylene glycolbis(2-aminoethyl)-N,N,N′,N′-tetraacetic acid (EGTA); 1,4,8,11-tetraazacyclotetradecane-1,4,8,11-tetraacetic acid (TETA); ethylenebis-(2-hydroxy-phenylglycine) (EHPG); 5-Cl-EHPG; 5-Br-EHPG; 5-Me-EHPG; 5t-Bu-EHPG; 5-sec-Bu-EHPG; benzodiethylenetriamine pentaacetic acid (benzo-DTPA); dibenzo-DTPA; phenyl-DTPA; diphenyl-DTPA; benzyl-DTPA; dibenzyl-DTPA; bis-2-(hydroxybenzyl)-ethylene-diaminediacetic acid (HBED) and derivatives thereof; Ac-DOTA; benzo-DOTA; dibenzo-DOTA; 1,4,7-triazacyclononane N,N′N″-triacetic acid (NOTA); benzo-NOTA; benzo-TETA; benzo-DOTMA, where DOTMA is 1,4,7,10-tetraazacyclotetradecane-1,4,7,10-tetra(methyltetraacetic acid); benzo-TETMA, where TETMA is 1,4,8,11-tetraazacyclotetradecane-1,4,8,11-(methyl tetraacetic acid); derivatives of 1,3-propylenediaminetetraacetic acid (PDTA); triethylenetetraaminehexaacetic acid (TTHA); derivatives of 1,5,10-N,N′,N″-tris(2,3-dihydrobenzoyl)-tricatecholate (LICAM); and 1,3,5-N,N′,N″-tris(2,3-dihydrobenzoyl)aminomethylbenzene (MECAM). In certain embodiments, the metal-ion chelator comprises a moiety represented by the structure:

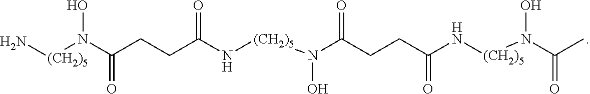

In certain embodiments, the labeling molecule comprises DFO, NOTA or DOTA as the metal ion chelator. In certain embodiments, the labeling molecule comprises DIBO as the reactive group. In certain embodiments, the labeling molecule comprises DIBO as the reactive group and DFO as the metal ion chelator (herein denoted as “DIBO-DFO”).

In certain embodiments, the labeling molecule comprises a tyrosine moiety, a reactive group, and a fluorophore. In certain embodiments, ¹²⁵I can be used as the radioactive ion when the labeling molecule comprises a tyrosine moiety.

In certain embodiments, the fluorophore is selected from the group consisting of a coumarin, a cyanine, a benzofuran, a quinolone, a quinazoline, an indole, a benzazole, a borapolyazaindacine, and a xanthene, which includes a fluorescein, a rhodamine, and a rhodol.

In certain embodiments, step (c) is performed in a solution substantially free of proteases. In certain embodiments, the radioactive metal ion is selected from the group consisting of ⁴⁵Ti, ⁵¹Mn, ⁵²mMn, ⁵²mMn, ⁵²Fe, ⁶⁰Cu, ⁶¹Cu, ⁶⁴Cu, ⁶⁷Cu, ⁶⁷Ga, ⁶⁸Ga, ⁷²As, ⁸⁹Y, ⁸⁹Zr, ⁹⁴mTc, 99mTc, ¹¹⁰In, ¹¹¹In, ¹¹³In, and ¹⁷⁷Lu.

In certain embodiments, methods are provided for radiolabeling an antibody, the methods comprising:

a) providing an antibody comprising an oligosaccharide having a Gal-GlcNAc linkage;

b) providing a β-galactosidase which cleaves a Gal-GlcNAc linkage;

c) contacting the antibody with the β-galactosidase to provide an antibody comprising a terminal GlcNAc residue;

d) providing a UDP-GalNAz;

e) providing a galactosyl transferase Y289L mutant;

f) contacting the antibody comprising the terminal GlcNAc residue with the UDP-GalNAz and the galactosyl transferase Y289L mutant, wherein the GalNAz group of the UDP-GalNAz attaches to the terminal GlcNAc residue to provide a modified antibody;

g) providing a DIBO-DFO labeling molecule;

h) contacting the modified antibody with the DIBO-DFO labeling molecule, wherein the DIBO-DFO labeling molecule attaches to the GalNAz group to provide a labeled antibody;

i) providing a radioactive metal ion; and

j) contacting the labeled antibody with the radioactive metal ion, wherein the metal ion associates with the DIBO-DFO to provide a radiolabeled antibody.

In certain embodiments, the DIBO-DFO labeling molecule further comprises a fluorophore.

In certain embodiments, methods are provided for dual-labeling a glycoprotein, the methods comprising

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first labeled glycoprotein;

f) providing a second labeling molecule comprising a fluorophore and a reactive group;

g) contacting the first labeled glycoprotein with the second labeling molecule, wherein the reactive group of the second labeling molecule attaches to the chemical handle to provide a dual-labeled glycoprotein;

h) providing a radioactive metal ion; and

i) contacting the dual-labeled glycoprotein with the radioactive metal ion, wherein the metal ion associates with the chelator group to provide a radiolabeled, dual-labeled glycoprotein.

In certain embodiments, the reactive group of the first labeling molecule and the reactive group of the second labeling molecule are the same. In certain embodiments, the reactive group of the first labeling molecule and the reactive group of the second labeling molecule are different.

In certain embodiments, the first labeling molecule is added before the second labeling molecule. In certain embodiments, the second labeling molecule is added before the first labeling molecule. In certain embodiments, the first and second labeling molecules are added simultaneously.

In certain embodiments, the labeling molecule comprises a reactive group and a metal ion chelator. In certain embodiments, the labeling molecule comprises a reactive group that comprises a cyclooctyne. In certain embodiments, the labeling molecule comprises a DFO, a NOTA or a DOTA as the metal ion chelator. In certain embodiments, the labeling molecule comprises a DIBO molecule and a DFO molecule. In certain embodiments, the labeling molecule comprises reactive group and a fluorophore. In certain embodiments, the fluorophore is selected from a xanthene, a cyanine, or a borapolyazaindacine. In certain embodiments, the labeling molecule comprises a DIBO molecule and a xanthene fluorophore. In certain embodiments, the labeling molecule comprises a DIBO molecule and a cyanine fluorophore.

In certain embodiments, the dual-labeled glycoprotein comprises an average fluorophore degree of labeling (DOL) of between about 0.1 and 5.0, between about 0.5 and 4.0, between about 1.0 and 3.0, between about 1.0 and 2.0, between about 1.0 and 1.5, or between about 2.0 and 2.5. In certain embodiments, the dual-labeled glycoprotein comprises an average metal ion chelator DOL of between about 0.1 and 5.0, between about 0.5 and 4.0, between about 1.0 and 3.0, between about 1.0 and 2.0, between about 1.0 and 1.5, or between about 2.0 and 2.5. In certain embodiments, the dual-labeled glycoprotein comprises an average fluorophore DOL of between about 0.1 and about 5.0, and an average metal ion chelator DOL of between about 5.0 and about 0.1. In certain embodiments, the fluorophore DOL is between about 0.5 and about 4.0 and the chelator DOL is between about 4.0 and about 0.5. In certain embodiments, the fluorophore DOL is between about 1.0 and about 3.0 and the chelator DOL is between about 3.0 and about 1.0. In certain embodiments, the fluorophore DOL is between about 1.0 and about 2.0 and the chelator DOL is between about 2.0 and about 1.0. In certain embodiments, the fluorophore DOL is between about 1.0 and about 1.5 and the chelator DOL is between about 2.5 and about 2.0. In certain embodiments, the fluorophore DOL is between about 2.0 and about 2.5 and the chelator DOL is between about 1.5 and about 1.0.

In certain embodiments, the glycoprotein comprises an antibody or an Fc-fusion protein. In certain embodiments, the antibody is an IgA, an IgD, and IgE, an IgG, an IgM, or an IgY. In certain embodiments, the antibody has an affinity for a cell-associated antigen.

In certain embodiments, the terminal GlcNAc residues are naturally-occurring terminal GlcNAc residues.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, prior to step (f), the method further comprises the steps of contacting the first-labeled glycoprotein with an enzyme to provide a first-labeled glycoprotein comprising a terminal GlcNAc residue; providing a second modified sugar comprising a chemical handle; and contacting the first labeled glycoprotein with the second modified sugar, wherein the second modified sugar attaches to the terminal GlcNAc residue to provide a modified first labeled glycoprotein. In certain embodiments, the enzyme is an endoglycosidase, a sialidase, or a β-galactosidase. In certain embodiments, the modified sugars are the same. In certain embodiments, the modified sugars are different.

In certain embodiments, methods are provided for dual-labeling a glycoprotein, the methods comprising

a) providing a glycoprotein comprising a terminal GlcNAc residue;

b) providing a first modified sugar comprising a chemical handle;

c) contacting the glycoprotein with the first modified sugar, wherein the first modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first labeled glycoprotein;

f) contacting the first labeled glycoprotein with an enzyme to provide a first labeled glycoprotein comprising a terminal GlcNAc residue;

g) providing a second modified sugar comprising a chemical handle;

h) contacting the first labeled glycoprotein with the modified sugar, wherein the modified sugar attaches to the terminal GlcNAc residue to provide a modified first labeled glycoprotein;

i) providing a second labeling molecule comprising a fluorophore and a reactive group;

j) contacting the modified first labeled glycoprotein with the second labeling molecule, wherein the reactive group of the second labeling molecule attaches to the chemical handle to provide a dual-labeled glycoprotein;

k) providing a radioactive metal ion; and

l) contacting the dual-labeled glycoprotein with the radioactive metal ion, wherein the metal ion associates with the chelator group to provide a radiolabeled, dual-labeled glycoprotein.

In certain embodiments, the reactive group of the first labeling molecule and the reactive group of the second labeling molecule are the same. In certain embodiments, the reactive group of the first labeling molecule and the reactive group of the second labeling molecule are different. In certain embodiments, the first modified sugar and the second modified sugar are the same. In certain embodiments, the first modified sugar and the second modified sugar are different.

In certain embodiments, the modified sugar is attached to the terminal GlcNAc residue by a galactosyl transferase. In certain embodiments, the galactosyl transferase is a mutant galactosyl transferase. In certain embodiments, the galactosyl transferase is a Y289L mutant galactosyl transferase.

In certain embodiments, the chemical handle comprises an azide group, and the reactive group comprises a terminal triarylphosphine, an alkyne, a terminal alkyne, or an activated alkyne group. In certain embodiments, the chemical handle comprises a terminal triarylphosphine, terminal alkyne or activated alkyne group, and the reactive group comprises an azide group. In certain embodiments, the activated alkyne group comprises a cyclooctyne group, a difluorocyclooctyne group, a dibenzocyclooctyne group an aza-dibenzocyclooctyne group, or a cyclononyne group. In certain embodiments, the activated alkyne group comprises a dibenzocyclooctyne group. In certain embodiments, the dibenzocyclooctyne group is 4-dibenzocyclooctynol (DIBO). In certain embodiments, the chemical handle comprises a Diels-Alder diene and the reactive group comprises a Diels-Alder dienophile. In certain embodiments, the chemical handle comprises a Diels-Alder dienophile and the reactive group comprises a Diels-Alder diene. In certain embodiments, the chemical handle comprises a straight chain or branched chain C₁-C₁₂ carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR₃NH₂ (carbazide), —NR¹NR²(C═S)NR³NH₂ (thiocarbazide), or —ONH₂ (aminooxy), wherein each R¹, R² and R³ is independently H or alkyl having 1-6 carbons. In certain embodiments, the modified sugar comprising a chemical handle is UDP-GalNAz. In certain embodiments, the modified sugar comprising a chemical handle is UDP-GalKyne. In certain embodiments, the modified sugar comprising a chemical handle is UDP-GalKetone.

In certain embodiments, methods are provided for detecting the presence of a cell-associated antigen in a sample, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group and a reactive group;

e) contacting the modified glycoprotein with the labeling molecule, wherein the reactive group attaches to the chemical handle to provide a labeled glycoprotein;

f) providing a radioactive metal ion;

g) contacting the labeled glycoprotein with the radioactive metal ion, wherein the metal ion associates with the chelator group to provide a radiolabeled glycoprotein;

h) providing a sample;

i) contacting the sample with the radiolabeled glycoprotein; and

j) detecting the radioactive emission of the radiolabeled glycoprotein, wherein the emission detected correlates with the presence of the cell-associated antigen in the sample.

In certain embodiments, the labeling molecule further comprises a fluorophore.

In certain embodiments, methods are provided for detecting the presence of a cell-associated antigen in a sample, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group, a reactive group, and a fluorophore;

e) contacting the modified glycoprotein with the labeling molecule, wherein the reactive group attaches to the chemical handle to provide a labeled glycoprotein;

f) providing a radioactive metal ion;

g) contacting the labeled glycoprotein with the radioactive metal ion, wherein the metal ion associates with the chelator group to provide a radiolabeled glycoprotein;

h) providing a sample;

i) contacting the sample with the radiolabeled glycoprotein; and

j) detecting the radioactive emission and/or the fluorescence emission of the radiolabeled glycoprotein, wherein the emission detected correlates with the presence of the cell-associated antigen in the sample.

In certain embodiments, the glycoprotein comprises an antibody or an Fc-fusion protein. In certain embodiments, the antibody is an IgA, an IgD, an IgE, an IgG, an IgM, or an IgY. In certain embodiments, the antibody has an affinity for a cell-associated antigen.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, the modified sugar is attached to the terminal GlcNAc residue by a galactosyl transferase. In certain embodiments, the galactosyl transferase is a mutant galactosyl transferase. In certain embodiments, the galactosyl transferase is a Y289L mutant galactosyl transferase.

In certain embodiments, the chemical handle comprises an azide group, and the reactive group comprises a terminal triarylphosphine, an alkyne, a terminal alkyne, or an activated alkyne group. In certain embodiments, the chemical handle comprises a terminal triarylphosphine, an alkyne, a terminal alkyne or an activated alkyne group, and the reactive group comprises an azide group. In certain embodiments, the activated alkyne group comprises a cyclooctyne group, a difluorocyclooctyne group, a dibenzocyclooctyne group, an aza-dibenzocyclooctyne group, or a cyclononyne group. In certain embodiments, the activated alkyne group comprises a dibenzocyclooctyne group. In certain embodiments, the dibenzocyclooctyne group is 4-dibenzocyclooctynol (DIBO). In certain embodiments, the chemical handle comprises a Diels-Alder diene and the reactive group comprises a Diels-Alder dienophile. In certain embodiments, the chemical handle comprises a Diels-Alder dienophile and the reactive group comprises a Diels-Alder diene. In certain embodiments, the chemical handle comprises a straight chain or branched chain C₁-C12 carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR³NH₂ (carbazide), —NR¹NR²(C═S)NR³NH₂ (thiocarbazide), or —ONH₂ (aminooxy), wherein each R¹, R² and R³ is independently H or alkyl having 1-6 carbons. In certain embodiments, the modified sugar comprising a chemical handle is UDP-GalNAz. In certain embodiments, the modified sugar comprising a chemical handle is UDP-GalKyne. In certain embodiments, the modified sugar comprising a chemical handle is UDP-GalKetone.

In certain embodiments, the sample is selected from the group consisting of a subject, a tissue from a subject, a cell from a subject, and a bodily fluid from a subject. In certain embodiments, the subject is a mammal. In certain embodiments, the detection of the radioactive emission is performed by positron emission tomography (PET). In certain embodiments, the detection of the radioactive emission is performed by single photon emission computer tomography (SPECT).

In certain embodiments, methods are provided for detecting the presence of a cell-associated antigen in a subject, the methods comprising the steps of:

a) providing an antibody comprising an oligosaccharide having a Gal-GlcNAc linkage and which recognizes the cell-associated antigen;

b) providing a β-galactosidase which cleaves a Gal-GlcNAc linkage;

c) contacting the antibody with the β-galactosidase to provide an antibody comprising a terminal GlcNAc residue;

d) providing a UDP-GalNAz;

e) providing a galactosyl transferase Y289L mutant;

f) contacting the antibody comprising the terminal GlcNAc residue with the UDP-GalNAz and the galactosyl transferase Y289L mutant, wherein the GalNAz group of the UDP-GalNAz attaches to the terminal GlcNAc residue to provide a modified antibody;

g) providing a DIBO-DFO labeling molecule;

h) contacting the modified antibody with the DIBO-DFO labeling molecule, wherein the DIBO-DFO labeling molecule attaches to the GalNAz group to provide a labeled antibody;

i) providing a radioactive metal ion;

j) contacting the labeled antibody with the radioactive metal ion, wherein the metal ion associates with the chelator group to provide a radiolabeled antibody;

k) providing a subject;

l) administering the radiolabeled antibody to the subject; and

m) detecting the radioactive emission of the radiolabeled antibody, wherein the emission detected correlates with the presence of the cell-associated antigen in the subject.

In certain embodiments, the DIBO-DFO labeling molecule further comprises a fluorophore.

In certain embodiments, methods are provided for detecting the presence of a cell-associated antigen in a sample, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first-labeled glycoprotein;

f) providing a second labeling molecule comprising a fluorophore and a reactive group;

g) contacting the first labeled glycoprotein with the second labeling molecule, wherein the reactive group of the second labeling molecule attaches to the chemical handle to provide a dual-labeled glycoprotein;

h) providing a radioactive metal ion;

i) contacting the dual-labeled glycoprotein with the radioactive metal ion, wherein the metal ion associates with the chelator group to provide a radiolabeled, dual-labeled glycoprotein;

j) providing a sample;

k) contacting the sample with the radiolabeled, dual-labeled glycoprotein; and

l) detecting the radioactive emission and/or the fluorescence emission of the radiolabeled, dual-labeled glycoprotein, wherein the emission detected correlates with the presence of the cell-associated antigen in the sample.

In certain embodiments, the first labeling molecule is added before the second labeling molecule. In certain embodiments, the second labeling molecule is added before the first labeling molecule. In certain embodiments, the first and second labeling molecules are added simultaneously. In certain embodiments, the reactive group of the first labeling molecule and the reactive group of the second labeling molecule are the same. In certain embodiments, the reactive group of the first labeling molecule and the reactive group of the second labeling molecule are different.

In certain embodiments, the glycoprotein comprises an antibody or an Fc-binding protein. In certain embodiments, the antibody may be an IgA, an IgD, an IgE, an IgG, an IgM, or an IgY. In certain embodiments, the antibody has an affinity for a cell-associated antigen.

In certain embodiments, the terminal GlcNAc residues are naturally-occurring terminal GlcNAc residues.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, prior to step (f), the method further comprises the steps of contacting the first-labeled glycoprotein with an enzyme to provide a first-labeled glycoprotein comprising a terminal GlcNAc residue; providing a second modified sugar comprising a chemical handle; and contacting the first labeled glycoprotein with the second modified sugar, wherein the second modified sugar attaches to the terminal GlcNAc residue to provide a modified first labeled glycoprotein. In certain embodiments, the enzyme is an endoglycosidase, a sialidase, or a β-galactosidase. In certain embodiments, the modified sugars are the same. In certain embodiments, the modified sugars are different.

In certain embodiments, methods are provided for detecting the presence of a cell-associated antigen in a sample, the methods comprising:

a) providing a glycoprotein comprising a terminal GlcNAc residue;

b) providing a first modified sugar comprising a chemical handle;

c) contacting the glycoprotein with the first modified sugar, wherein the first modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first-labeled glycoprotein;

f) contacting the first labeled glycoprotein with an enzyme to provide a first labeled glycoprotein comprising a terminal GlcNAc residue;

g) providing a second modified sugar comprising a chemical handle;

h) contacting the first labeled glycoprotein with the second modified sugar, wherein the modified sugar attaches to the terminal GlcNAc residue to provide a modified first labeled glycoprotein;

i) providing a second labeling molecule comprising a fluorophore and a reactive group;

g) contacting the modified first labeled glycoprotein with the second labeling molecule, wherein the reactive group of the second labeling molecule attaches to the chemical handle to provide a dual-labeled glycoprotein;

k) providing a radioactive metal ion;

l) contacting the dual-labeled glycoprotein with the radioactive metal ion, wherein the metal ion associates with the chelator group to provide a radiolabeled, dual-labeled glycoprotein;

m) providing a sample;

n) contacting the sample with the radiolabeled, dual-labeled glycoprotein; and

o) detecting the radioactive emission and/or the fluorescence emission of the radiolabeled, dual-labeled glycoprotein, wherein the emission detected correlates with the presence of the cell-associated antigen in the sample.

In certain embodiments, the reactive group of the first labeling molecule and the reactive group of the second labeling molecule are the same. In certain embodiments, the reactive group of the first labeling molecule and the reactive group of the second labeling molecule are different. In certain embodiments, the first modified sugar and the second modified sugar are the same. In certain embodiments, the first modified sugar and the second modified sugar are different.

Certain embodiments provide for the use of any of the methods, compositions or kits disclosed herein for the diagnosis of diseases, for example, cancer, including but not limited to breast cancer, prostate cancer, lung cancer, skin cancer, cancers of the reproductive tract, brain cancer, liver cancer, pancreatic cancer, stomach cancer, blood cancers (e.g., leukemia and lymphoma), sarcomas, melanomas, and the like.

Certain embodiments provide for the use of any of the methods, compositions or kits disclosed herein for the treatment of diseases, for example, cancer, including but not limited to breast cancer, prostate cancer, lung cancer, skin cancer, cancers of the reproductive tract, brain cancer, liver cancer, pancreatic cancer, stomach cancer, blood cancers (e.g., leukemia and lymphoma), sarcomas, melanomas, and the like.

In another aspect, compositions are provided for use in the methods provided herein. In certain embodiments, the compositions comprise a labeling molecule that comprises a metal ion chelator and a reactive group. In certain embodiments, the labeling molecule further comprises a fluorophore. In certain embodiments, the labeling molecule comprises a metal ion chelator, a reactive group, and a fluorophore. In certain embodiments, the compositions comprise a labeling molecule that comprises a reactive group and a fluorophore. In certain embodiments, the compositions comprise a tyrosine, a fluorophore, and a reactive group. In certain embodiments, the compositions comprise a labeling molecule having Formula (I):

FLUOROPHORE-REACTIVE GROUP-METAL ION CHELATOR (I)

wherein,

FLUOROPHORE is a coumarin, a cyanine, a benzofuran, a quinolone, a quinazoline, an indole, a benzazole, a borapolyazaindacine, or a xanthene;

REACTIVE GROUP comprises a terminal triarylphosphine, an alkyne, a terminal alkyne, an activated alkyne group, an azide, a ketone, a hydrazide, a semicarbazide, a thiocarbonylhydrazide, a carbonylhydrazide, a thiocarbonylhydrazide, a sulfonylhydrazide, a carbazide, a thiocarbazide, or an aminooxy group, a Diels-Alder diene, a Diels-Alder dienophile; and

METAL ION CHELATOR is a1,4,8,11-tetraazabicyclo[6.6.2]hexadecane-4,11-diyl)diacetic acid (CB-TE2A); desferrioxamine; diethylenetriaminepentaacetic acid (DTPA); 1,4,7,10-tetraazacyclotetradecane-1,4,7,10-tetraacetic acid (DOTA); ethylenediaminetetraacetic acid (EDTA); ethylene glycolbis(2-aminoethyl)-N,N,N′,N′-tetraacetic acid (EGTA); 1,4,8,11-tetraazacyclotetradecane-1,4,8,11-tetraacetic acid (TETA); ethylenebis-(2-4hydroxy-phenylglycine) (EHPG); 5-Cl-EHPG; 5Br-EHPG; 5-Me-EHPG; 5t-Bu-EHPG; 5-sec-Bu-EHPG; benzodiethylenetriamine pentaacetic acid (benzo-DTPA); dibenzo-DTPA; phenyl-DTPA, diphenyl-DTPA; benzyl-DTPA; dibenzyl DTPA; bis-2 (hydroxybenzyl)-ethylene-diaminediacetic acid (HBED) and derivatives thereof; Ac-DOTA; benzo-DOTA; dibenzo-DOTA; NOTA (1,4,7-triazacyclononane N,N′,N”-triacetic acid); benzo-NOTA; benzo-TETA, benzo-DOTMA, where DOTMA is 1,4,7,10-tetraazacyclotetradecane-1,4,7,10-tetra(methyl tetraacetic acid), benzo-TETMA, where TETMA is 1,4,8,11-tetraazacyclotetradecane-1,4,8,11-(methyl tetraacetic acid); derivatives of 1,3-propylenediaminetetraacetic acid (PDTA); triethylenetetraaminehexaacetic acid (TTHA); derivatives of 1,5,10-N,N′,N″-tris(2,3-dihydroxybenzoyl)-tricatecholate (LICAM); and 1,3,5-N,N′,N″-tris(2,3-dihydroxybenzoyl)aminomethylbenzene (MECAM).

In certain embodiments, the composition comprises a labeling molecule of

Formula (I), wherein the fluorophore is selected from a xanthene, a cyanine, a borapolyazaindacine, and a coumarin; the reactive group is an activated alkyne group; and the metal ion chelator is selected from DFO, NOTA and DOTA.

In certain embodiments, the composition comprises a labeling molecule of Formula (I), wherein the fluorophore is selected from a xanthene, a cyanine, a borapolyazaindacine, and a coumarin; the reactive group is a cyclooctyne; and the metal ion chelator is selected from DFO, NOTA and DOTA.

In certain embodiments, the composition comprises a labeling molecule of Formula (I), wherein fluorophore is selected from a xanthene, a cyanine, a borapolyazaindacine, and a coumarin; the reactive group is a DIBO; and the metal ion chelator is selected from DFO, NOTA and DOTA.

In another aspect, kits are provided for use in the methods provided herein. In certain embodiments, kits are provided for labeling a glycoprotein that include a modified sugar comprising a chemical handle, and a labeling molecule comprising a metal ion chelator group and a reactive group. In certain embodiments, the kits further comprise instructions for using the components in any of the methods as described herein. In certain embodiments, kits are provided for dual-labeling a glycoprotein that include a modified sugar comprising a chemical handle, and a labeling molecule comprising a metal ion chelator group, a reactive group and a fluorophore. In certain embodiments, the kits further include instructions for using the components in any of the methods as described herein. In certain embodiments, kits are provided for dual-labeling a glycoprotein that include a modified sugar comprising a chemical handle, a first labeling molecule comprising a metal ion chelator and a reactive group, and a second labeling molecule comprising a fluorophore and a reactive group. In certain embodiments, the kits further include instructions for using the components in any of the methods as described herein. In certain embodiments, kits are provided for labeling glycoproteins comprising a modified sugar comprising a chemical handle, and a labeling molecule comprising a tyrosine group, a reactive group, and a fluorophore. In certain embodiments, the kits further include instructions for using the components in any of the methods described herein.

In certain embodiments, kits are provided for detecting a cell-associated antigen that include a modified sugar comprising a chemical handle, and a labeling molecule comprising a metal ion chelator group and a reactive group. In certain embodiments, the kits further include instructions for using the components in any of the methods as described herein. In certain embodiments, kits are provided for detecting a cell-associated antigen that include a modified sugar comprising a chemical handle, and a labeling molecule comprising a metal ion chelator group, a reactive group, and a fluorophore. In certain embodiments, the kits further include instructions for using the components in any of the methods as described herein. In certain embodiments, kits are provided for detecting a cell-associated antigen that include a modified sugar comprising a chemical handle, a first labeling molecule comprising a metal ion chelator and a reactive group, and a second labeling molecule comprising a fluorophore and a reactive group. In certain embodiments, the kits further include instructions for using the components in any of the methods as described herein. In certain embodiments, kits are provided for detecting a cell-associated antigen comprising a modified sugar comprising a chemical handle, and a labeling molecule comprising a tyrosine group, a reactive group, and a fluorophore. In certain embodiments, the kits further include instructions for using the components in any of the methods described herein.

In certain embodiments, the kits may further include one or more of the following: an endoglycosidase, a sialidase, a β-galactosidase, a galactosyl transferase, a mutant galactosyl transferase, a Y289L mutant galactosyl transferase, a glycoprotein, an antibody, an Fc-fusion protein, and a radioactive metal ion. In certain embodiments, the kits may further include one or more of the following: one or more buffers, detergents and/or solvents.

### DESCRIPTION OF THE DRAWINGS

FIG. 1: Schematic of a strategy for the site-specific, enzyme- and click chemistry-mediated radiolabeling of antibodies on the heavy chain glycans according to certain embodiments of the present disclosure.

FIG. 2: Two schematics (A) and (B) of labeling molecules according to certain embodiments of the present disclosure.

FIG. 3: SDS-PAGE of unmodified (lanes 1, 2), GalNAz-modified (lanes 3, 5), or DIBO-DFO-modified (lanes 4, 6) antibody constructs either untreated (lanes 1, 3, 4) or treated (lanes 2, 5, 6) with PNGase F.

FIG. 4: Biodistribution of ⁸⁹Zr-DFO-DIBO/GalNAz-J591 (A) and ⁸⁹Zr-DFO-NCS-J591 (B) (15-20 μCi, 4-6 μg) in athymic nude mice bearing subcutaneous, PSMA-expressing LNCaP prostate cancer xenografts.

FIG. 5: PET images of ⁸⁹Zr-DFO-DIBO GalNAz-J591 and ⁸⁹Zr-DFO-NCS-J591 in athymic nude mice bearing subcutaneous, PSMA-expressing LNCaP prostate cancer xenografts (white arrows).

FIG. 6: The determination of the DOL of GalNAz-tagged J591 using a fluorescent DIBO derivative. (A) Gels were imaged with FUJI FLA9000 for Alexa Fluor® 488 with an excitation of 473 nm and a 510LP filter (right panel), then stained with SYPRO® Ruby Protein Stain and imaged with an excitation of 473 nm and a 575LP filter (left panel). (B) The degree of labeling (DOL) of the antibody with Click-iT® DIBO-Alexa Fluor® 488 was determined to be 2.7±0.2 (n=3) using the ratio of the fluorescence intensity of Alexa Fluor® 488 to that of SYPRO® Ruby (quantitated with Multi-Gauge). Labeling of GalNAz-J591 with DIBO-DFO prevented >95% of dye incorporation.

### DETAILED DESCRIPTION

Herein are provided methods, compositions and kits for use in the site-specific labeling of glycoproteins comprising a combination of enzyme-mediated incorporation of modified sugars comprising a chemical handle and cycloaddition chemistry with a labeling molecule comprising a metal ion chelator group, a reactive group that attaches to the chemical handle of the modified sugar, and optionally, a fluorophore. In certain embodiments, the glycoprotein comprises a terminal GlcNAc residue. In certain embodiments, the glycoprotein is an antibody or an Fc-fusion protein. In certain embodiments, the antibody is an IgA, an IgD, an IgE, an IgG, an IgM, or an IgY. In certain embodiments, the antibody has an affinity for a cell-associated antigen. In certain embodiments, the terminal GlcNAc residue is present on the Fc region of the antibody.

Antibodies such as IgGs contain a conserved N-linked glycosylation site on the CH₂ domain of each heavy chain of the Fc region. N-linked oligosaccharides from a variety of different animal species show a heterogeneous mixture of biantennary complex-type oligosaccharides (Raju et al., Glycobiology 10:477-486 (2000)). Although heterogeneous with respect to their core fucose, sialic acid, and galactose monomers, the majority of these biantennary glycans are composed of the G0, G1, or G2 (i.e. 0, 1, or 2 terminal galactose residues, respectively) isoforms, with the specific ratio of isoforms dependent on species and physiological status. Because the glycans are located on the heavy chain Fc domain of the antibody, far from the antigen binding domains, they provide extremely attractive targets for site-selective chemical modification. An example of such a modification strategy relies upon the oxidation of vicinal alcohols on the sugar chains to aldehydes followed by subsequent labeling via reductive amination or hydrazide condensation reactions (Wolfe and Hage, Anal. Biochem. 231:123-130 (1995)). However, this method requires prolonged exposure of the antibody to low pH and harsh redox conditions and can result in non-selective modifications to amino acid side chains in the antibody. Unfortunately, this method can adversely affect the immunoreactivity of the antibody, and can defeat the purpose of the site-selective modification strategy entirely.

An alternative procedure for the site-selective modification of IgG heavy chain glycans utilizes a system based on unnatural UDP-sugar substrates and a substrate-permissive mutant of β-1,4-galactosyltransferase, GalT(Y289L) using bioorthogonal “click” chemistry (see, for example, Ramakrishnan and Qasba, J. Biol. Chem. 277:20833-20839 (2002) and Boeggeman et al., Bioconjugate Chem. 18:806-814 (2007)).

Importantly, however, while the copper-catalyzed azide-alkyne click reaction has been shown to be selective and efficient, the presence of both copper(I) and copper(II) can damage proteins and thus interfere with the structure and function of enzymes, fluorescent proteins, and antibodies. Furthermore, and more specific to the radiochemical applications, the Cu-catalyzed variant of this click reaction cannot be used in conjunction with radiometal chelators, as the presence of micromolar levels of Cu catalyst can interfere with the chelation chemistry of radiometals that are often present in extremely low concentrations. However, these limitations can be overcome by the strain-promoted azide-alkyne click reaction: a selective, bioorthogonal, and catalyst-free ligation between an azide and a strained cyclic alkyne such as dibenzocyclooctyne (Sletten and Bertozzi, Angew. Chem. Int. Ed. 48:6973-6998 (2009), Ning et al., Angew. Chem. Int. Ed. 47:2253-2255 (2008), and Laughlin et al., Science 320:664-667 (2008)). However, GalNAz has not been employed as a substrate for GalT(Y289L); rather, the hexosamine biosynthetic pathway has been used to metabolically tag O-GlcNAc-modified proteins with azides for subsequent labeling in vitro or in vivo (Agard and Bertozzi, Acc. Chem. Res. 42:788-797 (2009), Sletten and Bertozzi, supra, and Laughlin et al., supra). However, metabolic labeling of glycoproteins is not truly site-specific because it modifies both O- and N-linked glycans. In addition, the degree of labeling (DOL) is very low. The methods provided herein allow for a controlled labeling of specific N-linked glycans resulting in a higher DOL. Furthermore, the methods are much easier to use and have fewer steps than those previously described.

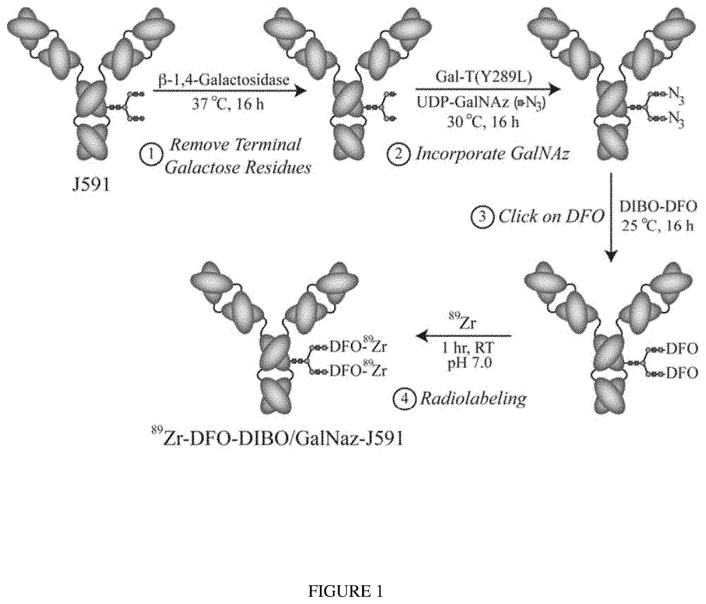

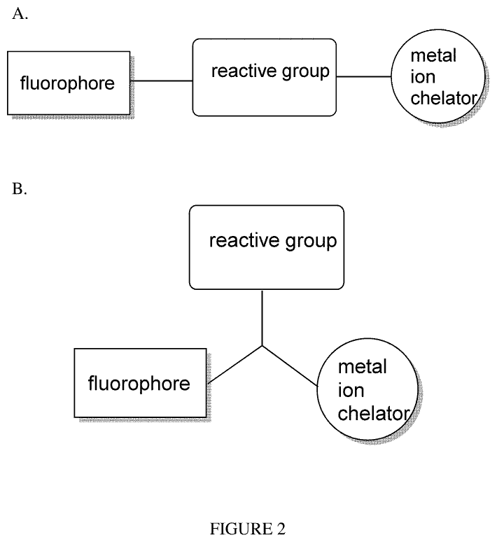

Herein are provided methods, compositions and kits for the site-selective radiolabeling of glycoproteins comprising a combination of both enzyme-mediated incorporation of modified sugars (such as GalNAz) and bioorthogonal, strain-promoted, copper-free azide/alkyne cycloaddition click chemistry. Generally, the methods described herein comprise: enzymatic removal of terminal galactose residues to expose terminal GlcNAc residues; enzymatic incorporation of GalNAz onto the terminal GlcNAc residues; catalyst-free, strain-promoted click conjugation of a novel chelator-modified cyclooctyne (such as DIBO) to the GalNAz; and radiolabeling of the chelator-modified construct with an appropriate radiometal (see, FIGS. 1 and 2). Because all antibodies possess N-linked glycans located only on the heavy chain Fc region, the methods provided herein are site-selective, and critically, unlike previous systems, requires no special antibody engineering. Further, the methods provided herein are mild, facile, highly reproducible, and the sites of labeling are easily and rapidly characterized. Taken together, this modular and robust labeling methodology may play a critical role in the development of novel radioimmunoconjugates and at the same time provide considerable time and cost savings by eliminating cumbersome optimization and characterization steps.

### Definitions:

The section headings used herein are for organizational purposes only and are not to be construed as limiting the desired subject matter in any way. All literature cited in the specification, including but not limited to, patents, patent applications, articles, books and treatises are expressly incorporated by reference in their entirety for any purpose. In the event that any of the incorporated literature contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Before describing the present teachings in detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise. Thus, for example, reference to “a ligand” includes a plurality of ligands and reference to “an antibody” includes a plurality of antibodies and the like.

Certain compounds disclosed herein can exist in unsolvated forms as well as solvated forms, including hydrated forms. In general, the solvated forms are equivalent to unsolvated forms and are encompassed within the scope of the present teachings.

Certain compounds disclosed herein may exist in multiple crystalline or amorphous forms. In general, all physical forms are equivalent for the uses contemplated by the present disclosure and are intended to be within the scope of the present teachings.

Certain compounds disclosed herein possess asymmetric carbon atoms (optical centers) or double bonds; the racemates, diastereomers, geometric isomers and individual isomers are encompassed within the scope of the present teachings.

The compounds described herein may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose an appropriate method for a particular situation. See, generally, Furniss et al. (eds.),VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), carbon-14 (¹⁴C), ⁴⁵Ti, ⁵¹Mn, ⁵²Mn, 52mMn, 52Fe, ⁶⁰Cu, ⁶¹Cu, ⁶⁴Cu, ⁶⁷Cu, ⁶⁷Ga, ⁶⁸Ga, ⁷²As, ⁸⁹Y, ⁸⁹Zr, ⁹⁴mTc, ⁹⁹mTc, ¹¹⁰In, ¹¹¹In, ¹¹³In, or ¹⁷⁷Lu. All isotopic variations of the compounds disclosed herein, whether radioactive or not, are intended to be encompassed within the scope of the present teachings.

Where a disclosed compound includes a conjugated ring system, resonance stabilization may permit a formal electronic charge to be distributed over the entire molecule. While a particular charge may be depicted as localized on a particular ring system, or a particular heteroatom, it is commonly understood that a comparable resonance structure can be drawn in which the charge may be formally localized on an alternative portion of the compound.

Selected compounds having a formal electronic charge may be shown without an appropriate biologically compatible counterion. Such a counterion serves to balance the positive or negative charge present on the compound. As used herein, a substance that is biologically compatible is not toxic as used, and does not have a substantially deleterious effect on biomolecules. Examples of negatively charged counterions include, among others, chloride, bromide, iodide, sulfate, alkanesulfonate, arylsulfonate, phosphate, perchlorate, tetrafluoroborate, tetraarylboride, nitrate and anions of aromatic or aliphatic carboxylic acids. Preferred counterions may include chloride, iodide, perchlorate and various sulfonates. Examples of positively charged counterions include, among others, alkali metal, or alkaline earth metal ions, ammonium, or alkylammonium ions.

Where substituent groups are specified by their conventional chemical formulae, written from left to right, they equally encompass the chemically identical substituents, which would result from writing the structure from right to left, e.g., —CH₂O— is intended to also recite —OCH₂—.

Unless defined otherwise, all technical and scientific terms used herein have the same meaning as commonly understood by one of ordinary skill in the art to which this invention is related. The following terms are defined for purposes of the present disclosure.

TABLE 1
List of Abbreviations

| Abbreviation | Term |
|---|---|
| Gal | Galactose |
| GalNAz | N-alpha-azidoacetylgalactosamine. |
| GlcNAz | N-alpha-azidoacetylglucosamine. |
| GalNAc | N-acetylgalactosamine. |
| GlcNAc | N-acetylglucosamine |
| NeuAc | N-acetylneuraminic acid |
| GalKyne | Alkyne-modified galactose |
| GalKetone | Ketone-modified galactose |

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C₁-C6 means one to six carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present teachings contain between about one and about twenty five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P, S, and Se and wherein the nitrogen, phosphorous, sulfur, and selenium atoms are optionally oxidized, and the nitrogen heteroatom is optionally be quaternized. The heteroatom(s) O, N, P, S, Si, and Se may be placed at any interior position of the heteroalkyl group or at the position at which the alkyl group is attached to the remainder of the molecule. Examples include, but are not limited to, —CH₂CH₂OCH₃, —CH₂CH₂NHCH₃, —CH₂CH₂N(CH₃)CH₃, —CH₂SCH₂CH₃, —CH₂CH₂S(O)CH₃, —CH₂CH₂S(O)₂CH₃, —CH═CHOCH₃, —Si(CH₃)₃, —CH₂CH═NOCH₃, and —CH═CHN(CH₃)CH₃. Up to two heteroatoms may be consecutive, such as, for example, —CH₂NHOCH₃ and —CH₂OSi(CH₃)₃. Similarly, the term “heteroalkylene” by itself or as part of another substituent means a divalent radical derived from heteroalkyl, as exemplified, but not limited by, —CH₂CH₂SCH₂CH₂— and —CH₂SCH₂CH₂NHCH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

Each of the above terms (e.g., “alkyl” and “heteroalkyl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═NOR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NRC(NR′R″)=NR′″, —S(O)R′, —S(O )₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups.

When a compound includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

As used herein, the term “heteroatom” includes oxygen (O), nitrogen (N), sulfur (S), phosphorus (P), silicon (Si), and selenium (Se).

The term “amino” or “amine group” refers to the group —NR′R″ (or N⁺RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions.

The term “activated alkyne,” as used herein, refers to a chemical moiety that selectively reacts with an azide reactive group on another molecule to form a covalent chemical bond between the activated alkyne group and the alkyne reactive group. Activated alkynes include, but are not limited to the cyclooctynes and difluorocyclooctynes described by Agard et al., J. Am. Chem. Soc., 126(46):15046-15047 (2004); the dibenzocyclooctynes described by Boon et al., PCT Publication No. WO 2009/067663 A1 (2009); and the aza-dibenzocyclooctynes described by Debets et al., Chem. Comm., 46:97-99 (2010). These dibenzocyclooctynes (including the aza-dibenzocyclooctynes) described above are collectively referred to herein as cyclooctyne groups. Activated alkynes also include cyclononynes described by Dommerholt et al., Angew. Chem. 122:9612-9615 (2010)).

The term “affinity,” as used herein, refers to the strength of the binding interaction of two molecules, such as an antibody and an antigen or a positively charged moiety and a negatively charged moiety. For bivalent molecules such as antibodies, affinity is typically defined as the binding strength of one binding domain for the antigen, e.g. one Fab fragment for the antigen. The binding strength of both binding domains together for the antigen is referred to as “avidity”. As used herein “high affinity” refers to a ligand that binds to an antibody having an affinity constant (Kₐ) greater than 10⁴ M⁻¹, typically 10⁵-10¹¹ M⁻¹; as determined by inhibition ELISA or an equivalent affinity determined by comparable techniques such as, for example, Scatchard plots or using Ka/dissociation constant, which is the reciprocal of the Kₐ.

The term “alkyne reactive,” as used herein, refers to a chemical moiety that selectively reacts with an alkyne modified group on another molecule to form a covalent chemical bond between the alkyne modified group and the alkyne reactive group. Examples of alkyne-reactive groups include, but are not limited to, azides. “Alkyne-reactive” can also refer to a molecule that contains a chemical moiety that selectively reacts with an alkyne group.

The term “antibody” as used herein refers to an immunoglobulin molecule or immunologically active portion thereof, i.e., an antigen-binding portion. Examples of immunologically active portions of immunoglobulin molecules include immunoglobulin molecules or fragments thereof that comprise the F(ab) region and a sufficient portion of the Fc region to comprise the oligosaccharide linkage site, for example, the asparagine-GlcNAc linkage site. An antibody sometimes is a polyclonal, monoclonal, recombinant (e.g., a chimeric or humanized), fully human, non-human (e.g., murine), or a single chain antibody. An antibody may have effector function and may fix complement, and may be coupled to a toxin or imaging agent. Antibodies may be endogenous, or polyclonal wherein an animal is immunized to elicit a polyclonal antibody response or by recombinant methods resulting in monoclonal antibodies produced from hybridoma cells or other cell lines. It is understood that the term “antibody” as used herein includes within its scope any of the various classes or sub-classes of immunoglobulin derived from any of the animals conventionally used. An antibody may be, for example, an IgA, an IgD, an IgE, an IgG, an IgM, or an IgY.

The term “antibody fragments,” as used herein, refers to fragments of antibodies that retain the principal selective binding characteristics of the whole antibody. Particular fragments are well-known in the art, for example, Fab, Fab′, and F(ab′)₂, which are obtained by digestion with various proteases, pepsin or papain, and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1 domains; (ii) the dAb fragment (Ward, et al., Nature 341:544 (1989)), which consists of a VH domain; (iii) isolated CDR regions; and (iv) single-chain Fv molecules (scFv) described above. In addition, arbitrary fragments can be made using recombinant technology that retains antigen-recognition characteristics.

The term “antigen,” as used herein, refers to a molecule that induces, or is capable of inducing, the formation of an antibody or to which an antibody binds selectively, including but not limited to a biological material. Antigen also refers to “immunogen”. The target-binding antibodies selectively bind an antigen, as such the term can be used herein interchangeably with the term “target”.

The term “anti-region antibody,” as used herein, refers to an antibody that was produced by immunizing an animal with a select region that is a fragment of a foreign antibody wherein only the fragment is used as the immunogen. Regions of antibodies include the Fc region, hinge region, Fab region, etc. Anti-region antibodies include monoclonal and polyclonal antibodies. The term “anti-region fragment” as used herein refers to a monovalent fragment that was generated from an anti-region antibody of the present invention by enzymatic cleavage.

The term “aqueous solution,” as used herein, refers to a solution that is predominantly water and retains the solution characteristics of water. Where the aqueous solution contains solvents in addition to water, water is typically the predominant solvent.

The term “azide reactive,” as used herein, refers to a chemical moiety that selectively reacts with an azido modified group on another molecule to form a covalent chemical bond between the azido modified group and the azide reactive group. Examples of azide-reactive groups include, but are not limited to, phosphines, including, but not limited to, triarylphosphines; alkynes, including, but not limited to terminal alkynes; cyclononynes; and cyclooctynes and difluorocyclooctynes as described by Agard et al., J. Am. Chem. Soc., 126 (46):15046-15047 (2004), dibenzocyclooctynes as described by Boon et al., PCT Publication No. WO 2009/067663 A1 (2009), and aza-dibenzocyclooctynes as described by Debets et al., Chem. Comm., 46:97-99 (2010). The various dibenzocyclooctynes described above are collectively referred to herein as cyclooctyne groups. “Azide-reactive” can also refer to a molecule that contains a chemical moiety that selectively reacts with an azido group.

The term “buffer,” as used herein, refers to a system that acts to minimize the change in acidity or basicity of the solution against addition or depletion of chemical substances.

The term, “chemical handle” as used herein refers to a specific functional group, such as an azide; an alkyne, including, but not limited to, a terminal alkyne, an activated alkyne, a cyclooctyne, and a cyclononyne; a phosphite; a phosphine, including, but not limited to a triarylphosphine; and the like. A chemical handle is a moiety that is rarely found in naturally-occurring biomolecules and is chemically inert towards biomolecules (e.g., native cellular components), but when reacted with an azide-reactive or alkyne-reactive group the reaction can take place efficiently under biologically relevant conditions (e.g., cell culture conditions, such as in the absence of excess heat or harsh reactants). Chemical handles also include a Diels Alder diene; a Diels Alder dienophile; ketones; a straight or branched C₁-C₁₂ carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂(sulfonylhydrazide), —NR¹NR²(C═O)NR³NH₂ (carbazide), —NR¹NR²(C═S)NRNH₂ (thiocarbazide), or —ONH₂ (aminooxy), wherein each R¹, R², and R³ is independently H or alkyl having 1-6 carbons.

The term “click chemistry,” as used herein, refers to the Huisgen cycloaddition or the 1,3-dipolar cycloaddition between an azide and an alkyne to form a 1,2,4-triazole. Such chemical reactions can use, but are not limited to, simple heteroatomic organic reactants and are reliable, selective, stereospecific, and exothermic.

The term “cycloaddition” as used herein refers to a chemical reaction in which two or more π (pi)-electron systems (e.g., unsaturated molecules or unsaturated parts of the same molecule) combine to form a cyclic product in which there is a net reduction of the bond multiplicity. In a cycloaddition, the π (pi) electrons are used to form new π (pi) bonds. The product of a cycloaddition is called an “adduct” or “cycloadduct”. Different types of cycloadditions are known in the art including, but not limited to, [3+2] cycloadditions and Diels-Alder reactions. [3+2] cycloadditions, which are also called 1,3-dipolar cycloadditions, occur between a 1,3-dipole and a dipolarophile and are typically used for the construction of five-membered heterocyclic rings. The term “[3+2] cycloaddition” also encompasses “copperless” [3+2] cycloadditions between azides and cyclooctynes and difluorocyclooctynes described by Agard et al., J. Am. Chem. Soc., 126 (46):15046-15047 (2004), the dibenzocyclooctynes described by Boon et al., PCT Publication No. WO 2009/067663 A1 (2009), and the aza-dibenzocyclooctynes described by Debets et al., Chem. Comm., 46:97-99 (2010).

The term “detectable response” as used herein refers to an occurrence of, or a change in, a signal that is directly or indirectly detectable either by observation or by instrumentation. The detectable response may be an occurrence of a signal wherein a fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

The term “detectably distinct” as used herein refers to a signal that is distinguishable or separable by a physical property either by observation or by instrumentation. For example, a fluorophore is readily distinguishable either by spectral characteristics or by fluorescence intensity, lifetime, polarization or photo-bleaching rate from another fluorophore in the sample, as well as from additional materials that are optionally present.

The term “directly detectable” as used herein refers to the presence of a material or the signal generated from the material is immediately detectable by observation, instrumentation, or film without requiring chemical modifications or additional substances.

The term “fluorophore” as used herein refers to a composition that is inherently fluorescent or demonstrates a change in fluorescence upon binding to a biological compound or metal ion, i.e., fluorogenic. Fluorophores may contain substitutents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a benzazole, a borapolyazaindacene and xanthenes including fluorescein, rhodamine and rhodol as well as other fluorophores described in RICHARD P. HAUGLAND, MOLECULAR PROBES HANDBOOK OF FLUORESCENT PROBES AND RESEARCH CHEMICALS (10^{th} edition, CD-ROM, September 2005), which is herein incorporated by reference in its entirety.

The term “glycoprotein,” as used herein, refers to a protein that has been glycosolated and those that have been enzymatically modified, in vivo or in vitro, to comprise a sugar group. Glycoproteins may also include modified sugar groups. Glycoproteins include, but are not limited to, antibodies.

The term “kit,” as used herein, refers to a packaged set of related components, typically one or more compounds or compositions.

The term “label,” as used herein, refers to a chemical moiety or protein that is directly or indirectly detectable (e.g. due to its spectral properties, conformation or activity) when attached to a target or compound and used in the present methods, including reporter molecules, solid supports and carrier molecules. The label can be directly detectable (fluorophore or radiolabel). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. Numerous labels are known by those of skill in the art and include, but are not limited to, particles, fluorophores, and other labels that are described in RICHARD P. HAUGLAND, MOLECULAR PROBES HANDBOOK OF FLUORESCENT PROBES AND RESEARCH PRODUCTS (10^{th} edition, CD-ROM, September 2005), supra.

The term “phosphine reactive” as used herein refers to a chemical moiety that selectively reacts via Staudinger ligation with a phosphine group, including but not limited to a triarylphosphine group, on another molecule to form a covalent chemical bond between the triarylphosphine group and the phosphine reactive group. Examples of phosphine reactive groups include, but are not limited to, an azido group.

The terms “protein” and “polypeptide” are used herein in a generic sense to include polymers of amino acid residues of any length. The term “peptide” is used herein to refer to polypeptides having less than 100 amino acid residues, typically less than 10 amino acid residues. The terms apply to amino acid polymers in which one or more amino acid residues are an artificial chemical analogue of a corresponding naturally occurring amino acid, as well as to naturally occurring amino acid polymers.

The term “purified” as used herein refers to a preparation of a protein that is essentially free from contaminating proteins that normally would be present in association with the protein, e.g., in a cellular mixture or milieu in which the protein or complex is found endogenously such as serum proteins or cellular lysate.

The term “sample” as used herein refers to any material that may contain an analyte or cell-associated antigen for detection or quantification. The sample may also include diluents, buffers, detergents, and contaminating species, debris and the like that are found mixed with the target. Illustrative examples include urine, sera, blood plasma, total blood, saliva, tear fluid, cerebrospinal fluid, secretory fluids from nipples and the like. Also included are solid, gel or sol substances such as mucus, body tissues, cells and the like suspended or dissolved in liquid materials such as buffers, extractants, solvents and the like. Typically, the sample is a live cell, a biological fluid that comprises endogenous host cell proteins, nucleic acid polymers, nucleotides, oligonucleotides, peptides and buffer solutions. The sample may also be a lysate isolated from a cell. The sample may be in an aqueous solution, a viable cell culture or immobilized on a solid or semi-solid surface such as a polyacrylamide gel, membrane blot or on a microarray. The sample may also be a subject, such as a mammal.

The term “Staudinger ligation” as used herein refers to a chemical reaction developed by Saxon and Bertozzi (E. Saxon and C. Bertozzi, Science, 287: 2007-2010 (2000)) that is a modification of the classical Staudinger reaction. The classical Staudinger reaction is a chemical reaction in which the combination of an azide with a phosphine or phosphite produces an aza-ylide intermediate, which upon hydrolysis yields a phosphine oxide and an amine. A Staudinger reaction is a mild method of reducing an azide to an amine; and triphenylphosphine is commonly used as the reducing agent. In a Staudinger ligation, an electrophilic trap (usually a methyl ester) is appropriately placed on the aryl group of a triarylphosphine (usually ortho to the phosphorus atom) and reacted with the azide, to yield an aza-ylide intermediate, which rearranges in aqueous media to produce a compound with amide group and a phosphine oxide function. The Staudinger ligation is so named because it ligates (attaches/covalently links) the two starting molecules together, whereas in the classical Staudinger reaction, the two products are not covalently linked after hydrolysis.

In general, for ease of understanding the present disclosure the methods for site-specific labeling of glycoproteins will first be described in detail. This will be followed by some embodiments in which such labeled glycoproteins can be used. Compositions and kits useful in the methods disclosed herein will also be discussed.

### “Click” Chemistry

Azides and terminal or internal alkynes can undergo a 1,3-dipolar cycloaddition (Huisgen cycloaddition) reaction to give a 1,2,3-triazole. However, this reaction requires long reaction times and elevated temperatures. Alternatively, azides and terminal alkynes can undergo Copper(I)-catalyzed Azide-Alkyne Cycloaddition (CuAAC) at room temperature. Such copper(I)-catalyzed azide-alkyne cycloadditions, also known as “click” chemistry, is a variant of the Huisgen 1,3-dipolar cycloaddition wherein organic azides and terminal alkynes react to give 1,4-regioisomers of 1,2,3-triazoles. Examples of “click” chemistry reactions are described by Sharpless et al. (U.S. Patent Application Publication No. 2005/0222427, published Oct. 6, 2005, International Application No. PCT /US03/17311; Lewis W G, et al., Angew. Chem. Int. Ed. 41 (6): 1053; method reviewed in Kolb, H. C., et al., Angew. Chem. Int. Ed. 40:2004-2021 (2001)), which developed reagents that react with each other in high yield and with few side reactions in a heteroatom linkage (as opposed to carbon-carbon bonds) in order to create libraries of chemical compounds.

The copper used as a catalyst for the “click” chemistry reaction to conjugate a label to a modified glycoprotein is in the Cu(I) reduction state. The sources of copper(I) used in such copper(I)-catalyzed azide-alkyne cycloadditions can be any cuprous salt including, but not limited to, cuprous halides such as cuprous bromide or cuprous iodide. However, this regioselective cycloaddition can also be conducted in the presence of a metal catalyst and a reducing agent. Copper can be provided in the Cu(II) reduction state (for example, as a salt, such as but not limited to Cu(NO₃)₂ Cu(OAc)₂ or CuSO₄), in the presence of a reducing agent wherein Cu(I) is formed in situ by the reduction of Cu(II). Such reducing agents include, but are not limited to, ascorbate, tris(2-carboxyethyl) phosphine (TCEP), NADH, NADPH, thiosulfate, metallic copper, hydroquinone, vitamin K₁, glutathione, cysteine, 2-mercaptoethanol, dithiothreitol, Fe²⁺, Co²⁺, or an applied electric potential. The reducing agents may also include metals selected from Al, Be, Co, Cr, Fe, Mg, Mn, Ni, Zn, Au, Ag, Hg, Cd, Zr, Ru, Fe, Co, Pt, Pd, Ni, Rh, and W.

Without limitation to any particular mechanism, copper in the Cu(I) state is a preferred catalyst for the copper(I)-catalyzed azide-alkyne cycloadditions, or “click” chemistry reactions. Certain metal ions, such as Cu(I), are unstable in aqueous solvents, therefore stabilizing ligands/chelators can be used to improve the reaction. Typically, at least one copper chelator is used, wherein such chelators bind copper in the Cu(I) state. Alternatively, at least one copper chelator is used, wherein such chelators bind copper in the Cu(II) state. In some instances, the copper(I) chelator is a 1,10 phenanthroline-containing copper (I) chelator. Non-limiting examples of such phenanthroline-containing copper (I) chelators include, but are not limited to, bathophenanthroline disulfonic acid (4,7-diphenyl-1,10-phenanthroline disulfonic acid) and bathocuproine disulfonic acid (BCS; 2,9-dimethyl-4,7-diphenyl-1,10-phenanthroline disulfonate). In other embodiments, the copper(I) chelator is THPTA as described in Jentzsch et al., Inorganic Chemistry, 48(2): 9593-9595 (2009), or the copper(I) chelators are those described in Finn et al., U.S. Patent Publication No. 2010/0197871. Other chelators used in such methods include, but are not limited to, N-(2-acetamido)iminodiacetic acid (ADA), pyridine-2,6-dicarboxylic acid (PDA), S-carboxymethyl-L-cysteine (SCMC), trientine, tetra-ethylenepolyamine (TEPA), N,N,N′,N′-tetrakis(2-pyridylmethyl)ethylenediamine (TPEN), EDTA, neocuproine, N-(2-acetamido)iminodiacetic acid (ADA), pyridine-2,6-dicarboxylic acid (PDA), S-carboxymethyl-L-cysteine (SCMC), tris-(benzyl-triazolylmethyl)amine (TBTA), or a derivative thereof. Most metal chelators, a wide variety of which are known in the chemical, biochemical, and medical arts, are known to chelate several metals, and thus metal chelators in general can be tested for their function in 1,3 cycloaddition reactions catalyzed by copper. Histidine may be used as a chelator, while glutathione may be used as a chelator and a reducing agent.

One or more copper chelators may be added more than once to such “click” chemistry reactions. In instances in which more than one copper chelator is added to a reaction, two or more of the copper chelators can bind copper in the Cu(I) state or, one or more of the copper chelators can bind copper in the Cu(I) state and one or more additional chelators can bind copper in the Cu(II) state.

### Activated Alkyne (“Copperless”) Chemistry

Azides and alkynes can undergo catalyst free [3+2] cycloaddition by a using the reaction of activated alkynes with azides. Such catalyst-free [3+2] cycloaddition can be used in methods described herein to conjugate a label to a modified glycoprotein. Alkynes can be activated by ring strain such as, by way of example only, eight-membered ring structures, or nine-membered, appending electron-withdrawing groups to such alkyne rings, or alkynes can be activated by the addition of a Lewis acid such as, by way of example only, Au(I) or Au(III). Alkynes activated by ring strain have been described, and has been referred to as “copperless” [3+2] cycloaddition. For example, the cyclooctynes and difluorocyclooctynes described by Agard et al., J. Am. Chem. Soc., 126 (46):15046-15047 (2004), the dibenzocyclooctynes described by Boon et al., PCT International Publication No. WO 2009/067663 A1 (2009), the aza-dibenzocyclooctynes described by Debets et al., Chem. Comm., 46:97-99 (2010), and the cyclononynes described by Dommerholt et al., Angew. Chem. 122:9612-9615 (2010)).

In certain embodiments of the methods described herein, the modified glycoprotein can possess an azide moiety, whereupon the labeling molecule possesses an activated alkyne moiety; while in other embodiments the modified glycoprotein can possess an activated alkyne moiety, and the labeling molecule possesses an azide moiety.

### Staudinger Ligation

The Staudinger reaction, which involves reaction between trivalent phosphorous compounds and organic azides (Staudinger et al. Helv. Chim. Acta 2:635 (1919)), has been used for a multitude of applications. (Gololobov et al. Tetrahedron 37:437 (1980)); (Gololobov et al. Tetrahedron 48: 1353 (1992)). There are almost no restrictions on the nature of the two reactants. The Staudinger ligation is a modification of the Staudinger reaction in which an electrophilic trap (usually a methyl ester) is placed on a triaryl phosphine. In the Staudinger ligation, the aza-ylide intermediate rearranges, in aqueous media, to produce an amide linkage and the phosphine oxide, ligating the two molecules together, whereas in the Staudinger reaction the two products are not covalently linked after hydrolysis. Such ligations have been described in U.S. Patent Application No. 2006/0276658. In certain embodiments, the phosphine can have a neighboring acyl group such as an ester, thioester or N-acyl imidazole (i.e. a phosphinoester, phosphinothioester, phosphinoimidazole) to trap the aza-ylide intermediate and form a stable amide bond upon hydrolysis. In certain embodiments, the phosphine can be a di- or triarylphosphine to stabilize the phosphine. The phosphines used in the Staudinger ligation methods described herein to conjugate a label to a modified glycoprotein include, but are not limited to, cyclic or acyclic, halogenated, bisphosphorus, or even polymeric. Similarly, the azides can be alkyl, aryl, acyl or phosphoryl. In certain embodiments, such ligations are carried out under oxygen-free anhydrous conditions. The glycoproteins described herein can be modified using a Staudinger ligation.

In certain embodiments of the methods described herein, the modified glycoprotein can possess an azide moiety, whereupon the labeling molecule possesses a phosphine moiety, including, but not limited to, a triarylphosphine moiety; while in other embodiments the modified glycoprotein can possess the phosphine moiety, and the labeling molecule possesses an azide moiety.

### Methods of Labeling Glycoproteins:

Herein are provided methods, compositions and kits for use in the site-specific labeling of glycoproteins comprising a combination of enzyme-mediated incorporation of modified sugars comprising a chemical handle and cycloaddition chemistry with a labeling molecule comprising: a metal ion chelator group and a reactive group that attaches to the chemical handle of the modified sugar; a fluorophore and a reactive group that attaches to the chemical handle of the modified sugar; or a metal ion chelator, a reactive group that attaches to the chemical handle of the modified sugar, and a fluorophore. In certain embodiments, the glycoprotein comprises a terminal GlcNAc residue. In certain embodiments, the glycoprotein is an antibody or an Fc-fusion protein. In certain embodiments, the antibody is an IgA, an IgE, an IgD, an IgG, an IgM, or an IgY. In certain embodiments, the antibody has an affinity for a cell-associated antigen. In certain embodiments, the terminal GlcNAc residues are present on the Fc region of the antibody.

In certain embodiments, methods for labeling a glycoprotein are provided, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group and a reactive group;

e) contacting the modified glycoprotein with the labeling molecule, wherein the reactive group attaches to the chemical handle to provide a labeled glycoprotein;

f) providing a radioactive metal ion; and

g) contacting the labeled glycoprotein with the radioactive metal ion, wherein the metal ion associates with the chelator group to provide a radiolabeled glycoprotein.

In certain embodiments, the labeling molecule further comprises a fluorophore. In certain embodiments, the glycoprotein comprises an antibody or an Fc-fusion protein. In certain embodiments, the antibody is an IgA, an IgE, an IgD, an IgG, an IgM, or an IgY. In certain embodiments, the antibody has an affinity for a cell-associated antigen.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, the modified sugar is attached to the terminal GlcNAc residue by a galactosyl transferase. In certain embodiments, the galactosyl transferase is a mutant galactosyl transferase. In certain embodiments, the galactosyl transferase is a Y289L mutant galactosyl transferase.

In certain embodiments, methods for labeling a glycoprotein are provided, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group, a reactive group, and a fluorophore;

e) contacting the modified glycoprotein with the labeling molecule, wherein the reactive group attaches to the chemical handle to provide a labeled glycoprotein;

f) providing a radioactive metal ion; and

g) contacting the labeled glycoprotein with the radioactive metal ion, wherein the metal ion associates with the chelator group to provide a radiolabeled glycoprotein.

In certain embodiments, the glycoprotein comprises an antibody or an Fc-fusion protein. In certain embodiments, the antibody is an IgA, an IgE, an IgD, an IgG, an IgM, or an IgY. In certain embodiments, the antibody has an affinity for a cell-associated antigen.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, the chemical handle comprises an azide group, and the reactive group comprises a terminal triarylphosphine, an alkyne, a terminal alkyne, or an activated alkyne group. In certain embodiments, the chemical handle comprises a terminal triarylphosphine, an alkyne, a terminal alkyne or an activated alkyne group, and the reactive group comprises an azide group. In certain embodiments, the activated alkyne comprises a cyclooctyne group, a difluorocyclooctyne group, a dibenzocyclooctyne group, an aza-dibenzocyclooctyne group, or a cyclononyne group. In certain embodiments, the activated alkyne group comprises a dibenzocyclooctyne group. In certain embodiments, the dibenzocyclooctyne group is 4-dibenzocyclooctynol (DIBO). In certain embodiments, the chemical handle comprises a Diels-Alder diene and the reactive group comprises a Diels-Alder dienophile. In certain embodiments, the chemical handle comprises a Diels-Alder dienophile and the reactive group comprises a Diels-Alder diene. In certain embodiments, the chemical handle comprises a straight chain or branched chain C₁-C12 carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR³NH₂ (carbazide), —NR¹NR²(C═S)NR³NH₂ (thiocarbazide), or —ONH₂ (aminooxy), wherein each R¹, R² and R³ is independently H or alkyl having 1-6 carbons. In certain embodiments, the modified sugar comprising a chemical handle is UDP-GalNAz. In certain embodiments, the modified sugar comprising a chemical handle is UDP-GalKyne. In certain embodiments, the modified sugar comprising a chemical handle is UDP-GalKetone.

In certain embodiments, the metal chelating group is selected from the group consisting of a metal chelating dimer, a metal chelating trimer, a metal chelating oligomer, and a metal chelating polymer. In certain embodiments, the metal ion chelator group comprises a group selected from the group consisting of 1,4,8,11-tetraazabicyclo[6.6.2]hexadecane-4,11-diyl)diacetic acid (CB-TE2A); desferrioxamine (DFO); diethylenetriaminepentaacetic acid (DTPA); 1,4,7,10-tetraazacyclotetradecane-1,4,7,10-tetraacetic acid (DOTA); ethylenediaminetetraacetic acid (EDTA); ethylene glycolbis(2-aminoethyl)-N,N,N′,N′-tetraacetic acid (EGTA); 1,4,8,11-tetraazacyclotetradecane-1,4,8,11-tetraacetic acid (TETA); ethylenebis-(2-hydroxy-phenylglycine) (EHPG); 5-Cl-EHPG; 5-Br-EHPG; 5-Me-EHPG; 5t-Bu-EHPG; 5-sec-Bu-EHPG; benzodiethylenetriamine pentaacetic acid (benzo-DTPA); dibenzo-DTPA; phenyl-DTPA; diphenyl-DTPA; benzyl-DTPA; dibenzyl-DTPA; bis-2-(hydroxybenzyl)-ethylene-diaminediacetic acid (HBED) and derivatives thereof; Ac-DOTA; benzo-DOTA; dibenzo-DOTA; 1,4,7-triazacyclononane N,N′N″-triacetic acid (NOTA); benzo-NOTA; benzo-TETA; benzo-DOTMA, where DOTMA is 1,4,7,10-tetraazacyclotetradecane-1,4,7,10-tetra(methyltetraacetic acid); benzo-TETMA, where TETMA is 1,4,8,11-tetraazacyclotetradecane-1,4,8,11-(methyl tetraacetic acid); derivatives of 1,3-propylenediaminetetraacetic acid (PDTA); triethylenetetraaminehexaacetic acid (TTHA); derivatives of 1,5,10-N,N′,N″-tris(2,3-dihydrobenzoyl)-tricatecholate (LICAM); and 1,3,5-N,N′,N″-tris(2,3-dihydrobenzoyl)aminomethylbenzene (MECAM). In certain embodiments, the metal-ion chelator comprises a moiety represented by the structure:

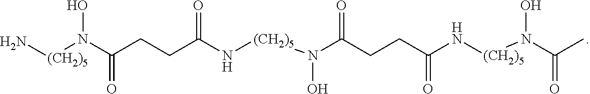

In certain embodiments, the labeling molecule comprises DFO, NOTA or DOTA as the metal ion chelator. In certain embodiments, the labeling molecule comprises DIBO as the reactive group. In certain embodiments, the labeling molecule comprises DIBO as the reactive group and DFO as the metal ion chelator (herein denoted as “DIBO-DFO”).

In certain embodiments, the labeling molecule comprises a tyrosine moiety, a reactive group, and a fluorophore. In certain embodiments, ¹²⁵I can be used as the radioactive ion when the labeling molecule comprises a tyrosine moiety.

In certain embodiments, the fluorophore is selected from the group consisting of a coumarin, a cyanine, a benzofuran, a quinolone, a quinazoline, an indole, a benzazole, a borapolyazaindacine, and a xanthene, which includes a fluorescein, a rhodamine, and a rhodol.

In certain embodiments, step (c) is performed in a solution substantially free of proteases. In certain embodiments, the radioactive metal ion is selected from the group consisting of ⁴⁵Ti, ⁵¹Mn, ⁵²Mn, ⁵²mMn, ⁵²Fe, ⁶⁰Cu, ⁶¹Cu, ⁶⁴Cu, ⁶⁷Cu, ⁶⁷Ga, ⁶⁸Ga, ⁷²As, ⁸⁹Y, ⁸⁹Zr, ⁹⁴mTc, ⁹⁹mTc, ¹¹⁰In, ¹¹¹In, ¹¹³In, and ¹⁷⁷Lu.

In certain embodiments, methods are provided for radiolabeling an antibody, the methods comprising:

a) providing an antibody comprising an oligosaccharide having a Gal-GlcNAc linkage;

b) providing a β-galactosidase which cleaves a Gal-GlcNAc linkage;

c) contacting the antibody with the β-galactosidase to provide an antibody comprising a terminal GlcNAc residue;

d) providing a UDP-GalNAz;

e) providing a galactosyl transferase Y289L mutant;

f) contacting the antibody comprising the terminal GlcNAc residue with the UDP-GalNAz and the galactosyl transferase Y289L mutant, wherein the GalNAz group of the UDP-GalNAz attaches to the terminal GlcNAc residue to provide a modified antibody;

g) providing a DIBO-DFO labeling molecule;

h) contacting the modified antibody with the DIBO-DFO labeling molecule, wherein the DIBO-DFO labeling molecule attaches to the GalNAz group to provide a labeled antibody;

i) providing a radioactive metal ion; and

j) contacting the labeled antibody with the radioactive metal ion, wherein the metal ion associates with the DIBO-DFO to provide a radiolabeled antibody.

In certain embodiments, the DIBO-DFO labeling molecule further comprises a fluorophore.

In certain embodiments, methods are provided for dual-labeling a glycoprotein, the methods comprising

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first labeled glycoprotein;

f) providing a second labeling molecule comprising a fluorophore and a reactive group;

g) contacting the first labeled glycoprotein with the second labeling molecule, wherein the reactive group of the second labeling molecule attaches to the chemical handle to provide a dual-labeled glycoprotein;

h) providing a radioactive metal ion; and

i) contacting the dual-labeled glycoprotein with the radioactive metal ion, wherein the metal ion associates with the chelator group to provide a radiolabeled, dual-labeled glycoprotein.

In certain embodiments, the reactive group of the first labeling molecule and the reactive group of the second labeling molecule are the same. In certain embodiments, the reactive group of the first labeling molecule and the reactive group of the second labeling molecule are different.

In certain embodiments, the first labeling molecule is added before the second labeling molecule. In certain embodiments, the second labeling molecule is added before the first labeling molecule. In certain embodiments, the first and second labeling molecules are added simultaneously.

In certain embodiments, the labeling molecule comprises a reactive group and a metal ion chelator. In certain embodiments, the labeling molecule comprises a reactive group that comprises a cyclooctyne. In certain embodiments, the labeling molecule comprises a DFO, a NOTA or a DOTA as the metal ion chelator. In certain embodiments, the labeling molecule comprises a DIBO molecule and a DFO molecule. In certain embodiments, the labeling molecule comprises reactive group and a fluorophore. In certain embodiments, the fluorophore is selected from a xanthene, a cyanine, or a borapolyazaindacine. In certain embodiments, the labeling molecule comprises a DIBO molecule and a xanthene fluorophore. In certain embodiments, the labeling molecule comprises a DIBO molecule and a cyanine fluorophore.

In certain embodiments, the dual-labeled glycoprotein comprises an average fluorophore degree of labeling (DOL) of between about 0.1 and 5.0, between about 0.5 and 4.0, between about 1.0 and 3.0, between about 1.0 and 2.0, between about 1.0 and 1.5, or between about 2.0 and 2.5. In certain embodiments, the dual-labeled glycoprotein comprises an average metal ion chelator DOL of between about 0.1 and 5.0, between about 0.5 and 4.0, between about 1.0 and 3.0, between about 1.0 and 2.0, between about 1.0 and 1.5, or between about 2.0 and 2.5. In certain embodiments, the dual-labeled glycoprotein comprises an average fluorophore DOL of between about 0.1 and about 5.0, and an average metal ion chelator DOL of between about 5.0 and about 0.1. In certain embodiments, the fluorophore DOL is between about 0.5 and about 4.0 and the chelator DOL is between about 4.0 and about 0.5. In certain embodiments, the fluorophore DOL is between about 1.0 and about 3.0 and the chelator DOL is between about 3.0 and about 1.0. In certain embodiments, the fluorophore DOL is between about 1.0 and about 2.0 and the chelator DOL is between about 2.0 and about 1.0. In certain embodiments, the fluorophore DOL is between about 1.0 and about 1.5 and the chelator DOL is between about 2.5 and about 2.0. In certain embodiments, the fluorophore DOL is between about 2.0 and about 2.5 and the chelator DOL is between about 1.5 and about 1.0.

In certain embodiments, the glycoprotein comprises an antibody or an Fc-fusion protein. In certain embodiments, the antibody is an IgA, an IgD, and IgE, an IgG, an IgM, or an IgY. In certain embodiments, the antibody has an affinity for a cell-associated antigen.

In certain embodiments, the terminal GlcNAc residues are naturally-occurring terminal GlcNAc residues.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, prior to step (f), the method further comprises the steps of contacting the first-labeled glycoprotein with an enzyme to provide a first-labeled glycoprotein comprising a terminal GlcNAc residue; providing a second modified sugar comprising a chemical handle; and contacting the first labeled glycoprotein with the second modified sugar, wherein the second modified sugar attaches to the terminal GlcNAc residue to provide a modified first labeled glycoprotein. In certain embodiments, the enzyme is an endoglycosidase, a sialidase, or a β-galactosidase. In certain embodiments, the modified sugars are the same. In certain embodiments, the modified sugars are different.

In certain embodiments, methods are provided for dual-labeling a glycoprotein, the methods comprising

a) providing a glycoprotein comprising a terminal GlcNAc residue;

b) providing a first modified sugar comprising a chemical handle;

c) contacting the glycoprotein with the first modified sugar, wherein the modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first labeled glycoprotein;

f) contacting the first labeled glycoprotein with an enzyme to provide a first labeled glycoprotein comprising a terminal GlcNAc residue;

g) providing a second modified sugar comprising a chemical handle;

h) contacting the first labeled glycoprotein with the modified sugar, wherein the modified sugar attaches to the terminal GlcNAc residue to provide a modified first labeled glycoprotein;

i) providing a second labeling molecule comprising a fluorophore and a reactive group;

j) contacting the modified first labeled glycoprotein with the second labeling molecule, wherein the reactive group of the second labeling molecule attaches to the chemical handle to provide a dual-labeled glycoprotein;

k) providing a radioactive metal ion; and

l) contacting the dual-labeled glycoprotein with the radioactive metal ion, wherein the metal ion associates with the chelator group to provide a radiolabeled, dual-labeled glycoprotein.

In certain embodiments, the reactive group of the first labeling molecule and the reactive group of the second labeling molecule are the same. In certain embodiments, the reactive group of the first labeling molecule and the reactive group of the second labeling molecule are different. In certain embodiments, the first modified sugar and the second modified sugar are the same. In certain embodiments, the first modified sugar and the second modified sugar are different.

In certain embodiments, the modified sugar is attached to the terminal GlcNAc residue by a galactosyl transferase. In certain embodiments, the galactosyl transferase is a mutant galactosyl transferase. In certain embodiments, the galactosyl transferase is a Y289L mutant galactosyl transferase.

In certain embodiments, the chemical handle comprises an azide group, and the reactive group comprises a terminal triarylphosphine, an alkyne, a terminal alkyne, or an activated alkyne group. In certain embodiments, the chemical handle comprises a terminal triarylphosphine, terminal alkyne or activated alkyne group, and the reactive group comprises an azide group. In certain embodiments, the activated alkyne group comprises a cyclooctyne group, a difluorocyclooctyne group, a dibenzocyclooctyne group an aza-dibenzocyclooctyne group, or a cyclononyne group. In certain embodiments, the activated alkyne group comprises a dibenzocyclooctyne group. In certain embodiments, the dibenzocyclooctyne group is 4-dibenzocyclooctynol (DIBO). In certain embodiments, the chemical handle comprises a Diels-Alder diene and the reactive group comprises a Diels-Alder dienophile. In certain embodiments, the chemical handle comprises a Diels-Alder dienophile and the reactive group comprises a Diels-Alder diene. In certain embodiments, the chemical handle comprises a straight chain or branched chain C₁-C12 carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR₃NH₂ (carbazide), —NR¹NR²(C═S)NR³NH₂ (thiocarbazide), or —ONH₂ (aminooxy), wherein each R¹, R² and R³ is independently H or alkyl having 1-6 carbons. In certain embodiments, the modified sugar comprising a chemical handle is UDP-GalNAz. In certain embodiments, the modified sugar comprising a chemical handle is UDP-GalKyne. In certain embodiments, the modified sugar comprising a chemical handle is UDP-GalKetone.

### Methods of Detection:

In certain embodiments, methods are provided for detecting the presence of a cell-associated antigen in a sample, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group and a reactive group;

e) contacting the modified glycoprotein with the labeling molecule, wherein the reactive group attaches to the chemical handle to provide a labeled glycoprotein;

f) providing a radioactive metal ion;

g) contacting the labeled glycoprotein with the radioactive metal ion, wherein the metal ion associates with the chelator group to provide a radiolabeled glycoprotein;

h) providing a sample;

i) contacting the sample with the radiolabeled glycoprotein; and

j) detecting the radioactive emission of the radiolabeled glycoprotein, wherein the emission detected correlates with the presence of the cell-associated antigen in the sample.

In certain embodiments, the labeling molecule further comprises a fluorophore.

In certain embodiments, methods are provided for detecting the presence of a cell-associated antigen in a sample, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a labeling molecule comprising a metal ion chelator group, a reactive group, and a fluorophore;

e) contacting the modified glycoprotein with the labeling molecule, wherein the reactive group attaches to the chemical handle to provide a labeled glycoprotein;

f) providing a radioactive metal ion;

g) contacting the labeled glycoprotein with the radioactive metal ion, wherein the metal ion associates with the chelator group to provide a radiolabeled glycoprotein;

h) providing a sample;

i) contacting the sample with the radiolabeled glycoprotein; and

j) detecting the radioactive emission and/or the fluorescence emission of the radiolabeled glycoprotein, wherein the emission detected correlates with the presence of the cell-associated antigen in the sample.

In certain embodiments, the glycoprotein comprises an antibody or an Fc-fusion protein. In certain embodiments, the antibody is an IgA, an IgD, an IgE, an IgG, an IgM, or an IgY. In certain embodiments, the antibody has an affinity for a cell-associated antigen.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, the modified sugar is attached to the terminal GlcNAc residue by a galactosyl transferase. In certain embodiments, the galactosyl transferase is a mutant galactosyl transferase. In certain embodiments, the galactosyl transferase is a Y289L mutant galactosyl transferase.

In certain embodiments, the chemical handle comprises an azide group, and the reactive group comprises a terminal triarylphosphine, an alkyne, a terminal alkyne, or an activated alkyne group. In certain embodiments, the chemical handle comprises a terminal triarylphosphine, an alkyne, a terminal alkyne or an activated alkyne group, and the reactive group comprises an azide group. In certain embodiments, the activated alkyne group comprises a cyclooctyne group, a difluorocyclooctyne group, a dibenzocyclooctyne group, an aza-dibenzocyclooctyne group, or a cyclononyne group. In certain embodiments, the activated alkyne group comprises a dibenzocyclooctyne group. In certain embodiments, the dibenzocyclooctyne group is 4-dibenzocyclooctynol (DIBO). In certain embodiments, the chemical handle comprises a Diels-Alder diene and the reactive group comprises a Diels-Alder dienophile. In certain embodiments, the chemical handle comprises a Diels-Alder dienophile and the reactive group comprises a Diels-Alder diene. In certain embodiments, the chemical handle comprises a straight chain or branched chain C₁-C12 carbon chain bearing a carbonyl group, and the reactive group comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR³NH₂ (carbazide), —NR¹NR²(C═S)NR³NH₂ (thiocarbazide), or —ONH₂ (aminooxy), wherein each R¹, R² and R³ is independently H or alkyl having 1-6 carbons. In certain embodiments, the modified sugar comprising a chemical handle is UDP-GalNAz. In certain embodiments, the modified sugar comprising a chemical handle is UDP-GalKyne. In certain embodiments, the modified sugar comprising a chemical handle is UDP-GalKetone.

In certain embodiments, the sample is selected from the group consisting of a subject, a tissue from a subject, a cell from a subject, and a bodily fluid from a subject. In certain embodiments, the subject is a mammal. In certain embodiments, the detection of the radioactive emission is performed by positron emission tomography (PET). In certain embodiments, the detection of the radioactive emission is performed by single photon emission computer tomography (SPECT).

In certain embodiments, methods are provided for detecting the presence of a cell-associated antigen in a subject, the methods comprising the steps of:

a) providing an antibody comprising an oligosaccharide having a Gal-GlcNAc linkage and which recognizes the cell-associated antigen;

b) providing a β-galactosidase which cleaves a Gal-GlcNAc linkage;

c) contacting the antibody with the β-galactosidase to provide an antibody comprising a terminal GlcNAc residue;

d) providing a UDP-GalNAz;

e) providing a galactosyl transferase Y289L mutant;

f) contacting the antibody comprising the terminal GlcNAc residue with the UDP-GalNA and the galactosyl transferase Y289L mutant, wherein the GalNAz group of the UDP-GalNAz attaches to the terminal GlcNAc residue to provide a modified antibody;

g) providing a DIBO-DFO labeling molecule;

h) contacting the modified antibody with the DIBO-DFO labeling molecule, wherein the DIBO-DFO labeling molecule attaches to the GalNAz group to provide a labeled antibody;

i) providing a radioactive metal ion;

j) contacting the labeled antibody with the radioactive metal ion, wherein the metal ion associates with the chelator group to provide a radiolabeled antibody;

k) providing a subject;

l) administering the radiolabeled antibody to the subject; and

m) detecting the radioactive emission of the radiolabeled antibody, wherein the emission detected correlates with the presence of the cell-associated antigen in the subject.

In certain embodiments, the DIBO-DFO labeling molecule further comprises a fluorophore.

In certain embodiments, methods are provided for detecting the presence of a cell-associated antigen in a sample, the methods comprising:

a) providing a glycoprotein comprising a terminal GlcNAc residue;

b) providing a modified sugar comprising a chemical handle;

c) contacting the glycoprotein with the modified sugar, wherein the modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first-labeled glycoprotein;

f) providing a second labeling molecule comprising a fluorophore and a reactive group;

g) contacting the first labeled glycoprotein with the second labeling molecule, wherein the reactive group of the second labeling molecule attaches to the chemical handle to provide a dual-labeled glycoprotein;

h) providing a radioactive metal ion;

i) contacting the dual-labeled glycoprotein with the radioactive metal ion, wherein the metal ion associates with the chelator group to provide a radiolabeled, dual-labeled glycoprotein;

j) providing a sample;

k) contacting the sample with the radiolabeled, dual-labeled glycoprotein; and

l) detecting the radioactive emission and/or the fluorescence emission of the radiolabeled, dual-labeled glycoprotein, wherein the emission detected correlates with the presence of the cell-associated antigen in the sample.

In certain embodiments, the first labeling molecule is added before the second labeling molecule. In certain embodiments, the second labeling molecule is added before the first labeling molecule. In certain embodiments, the first and second labeling molecules are added simultaneously. In certain embodiments, the reactive group of the first labeling molecule and the reactive group of the second labeling molecule are the same. In certain embodiments, the reactive group of the first labeling molecule and the reactive group of the second labeling molecule are different.

In certain embodiments, the glycoprotein comprises an antibody or an Fc-binding protein. In certain embodiments, the antibody may be an IgA, an IgD, an IgE, an IgG, an IgM, or an IgY. In certain embodiments, the antibody has an affinity for a cell-associated antigen.

In certain embodiments, the terminal GlcNAc residues are naturally-occurring terminal GlcNAc residues.

In certain embodiments, prior to step (c), the method further comprises the steps of providing a glycoprotein comprising an oligosaccharide having a GlcNAc-GlcNAc linkage; providing an enzyme to cleave the oligosaccharide at the GlcNAc-GlcNAc linkage; and contacting the glycoprotein with the enzyme to provide a glycoprotein comprising a terminal GlcNAc residue. In certain embodiments, the enzyme is an endoglycosidase.

In certain embodiments, prior to step (c), the method further comprises the steps of providing a glycoprotein comprising an oligosaccharide having a NeuAc-Gal-GlcNAc linkage; providing an enzyme to cleave the oligosaccharide at the NeuAc-Gal-GlcNAc linkage; and contacting the glycoprotein with the enzyme to provide a glycoprotein comprising an oligosaccharide having a Gal-GlcNAc linkage. In certain embodiments, the enzyme is a sialidase. In certain embodiments, the glycoprotein comprising the oligosaccharide having the Gal-GlcNAc linkage is further contacted with a second enzyme to cleave the oligosaccharide at the Gal-GlcNAc linkage to produce a glycoprotein comprising an oligosaccharide having a terminal GlcNAc residue. In certain embodiments, the second enzyme is a β-galactosidase.

In certain embodiments, prior to step (c), the method further comprises the steps of providing a glycoprotein comprising an oligosaccharide having a Gal-GlcNAc linkage; providing an enzyme to cleave the oligosaccharide at the Gal-GlcNAc linkage; and contacting the glycoprotein with the enzyme to provide a glycoprotein comprising an oligosaccharide having a terminal GlcNAc residue. In certain embodiments, the enzyme is a β-galactosidase.

In certain embodiments, prior to step (f), the method further comprises the steps of contacting the first-labeled glycoprotein with an enzyme to provide a first-labeled glycoprotein comprising a terminal GlcNAc residue; providing a second modified sugar comprising a chemical handle; and contacting the first labeled glycoprotein with the second modified sugar, wherein the second modified sugar attaches to the terminal GlcNAc residue to provide a modified first labeled glycoprotein. In certain embodiments, the enzyme is an endoglycosidase, a sialidase, or a β-galactosidase. In certain embodiments, the modified sugars are the same. In certain embodiments, the modified sugars are different.

In certain embodiments, methods are provided for detecting the presence of a cell-associated antigen in a sample, the methods comprising:

a) providing a glycoprotein comprising a terminal GlcNAc residue;

b) providing a first modified sugar comprising a chemical handle;

c) contacting the glycoprotein with the first modified sugar, wherein the modified sugar attaches to the terminal GlcNAc residue to provide a modified glycoprotein;

d) providing a first labeling molecule comprising a metal ion chelator group and a reactive group;

e) contacting the modified glycoprotein with the first labeling molecule, wherein the reactive group attaches to the chemical handle to provide a first-labeled glycoprotein;

f) contacting the first labeled glycoprotein with an enzyme to provide a first labeled glycoprotein comprising a terminal GlcNAc residue;

g) providing a second modified sugar comprising a chemical handle;

h) contacting the first labeled glycoprotein with the modified sugar, wherein the modified sugar attaches to the terminal GlcNAc residue to provide a modified first labeled glycoprotein;

i) providing a second labeling molecule comprising a fluorophore and a reactive group;

g) contacting the modified first labeled glycoprotein with the second labeling molecule, wherein the reactive group of the second labeling molecule attaches to the chemical handle to provide a dual-labeled glycoprotein;

k) providing a radioactive metal ion;

l) contacting the dual-labeled glycoprotein with the radioactive metal ion, wherein the metal ion associates with the chelator group to provide a radiolabeled, dual-labeled glycoprotein;

m) providing a sample;

n) contacting the sample with the radiolabeled, dual-labeled glycoprotein; and

o) detecting the radioactive emission and/or the fluorescence emission of the radiolabeled, dual-labeled glycoprotein, wherein the emission detected correlates with the presence of the cell-associated antigen in the sample.

In certain embodiments, the reactive group of the first labeling molecule and the reactive group of the second labeling molecule are the same. In certain embodiments, the reactive group of the first labeling molecule and the reactive group of the second labeling molecule are different. In certain embodiments, the first modified sugar and the second modified sugar are the same. In certain embodiments, the first modified sugar and the second modified sugar are different.

Certain embodiments provide for the use of any of the methods, compositions or kits disclosed herein for the diagnosis of diseases, for example, cancer, including but not limited to breast cancer, prostate cancer, lung cancer, skin cancer, cancers of the reproductive tract, brain cancer, liver cancer, pancreatic cancer, stomach cancer, blood cancers (e.g., leukemia and lymphoma), sarcomas, melanomas, and the like.

Certain embodiments provide for the use of any of the methods, compositions or kits disclosed herein for the treatment of diseases, for example, cancer, including but not limited to breast cancer, prostate cancer, lung cancer, skin cancer, cancers of the reproductive tract, brain cancer, liver cancer, pancreatic cancer, stomach cancer, blood cancers (e.g., leukemia and lymphoma), sarcomas, melanomas, and the like.

### Modification of Glycoproteins:

The glycoproteins that may be used in the methods disclosed herein may be any glycoprotein, including for example, hormones, enzymes, antibodies, Fc-fusion proteins, viral receptors, viral surface glycoproteins, parasite glycoproteins, parasite receptors, T-cell receptors, MHC molecules, immune modifiers, tumor antigens, mucins, inhibitors, growth factors, trophic factors, lymphokines, cytokines, toxoids, nerve growth hormones, blood clotting factors, adhesion molecules, multidrug resistance proteins, adenylate cyclases, bone morphogenic proteins and lectins. Additional glycoproteins contemplated for use in the methods disclosed herein include cross-linked glycoproteins, such as those described in U.S. Pat. No. 6,359,118, the contents of which are incorporated by reference. Preferably, the glycoproteins are antibodies or Fc-fusion proteins.

Antibodies for use in the methods disclosed herein may be produced using any means known to those of ordinary skill in the art. General information regarding procedures for antibody production and labeling may be found, for example, in Antibodies: A Laboratory Manual, Cold Spring Harbor Laboratory, Chap. 14 (1988). Cell lines expressing antibodies may also be produced using any means known to those of ordinary skill in the art. For therapeutic purposes, chimeric, humanized, and completely human antibodies are useful for applications that include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application No. 184,187; Taniguchi, M., European Patent Application Publication No. 171,496; Morrison et al European Patent Application Publication No. 173,494; Neuberger et al PCT International Publication No. WO 86/01533; Cabilly et al U.S. Patent No. 4,816,567; Cabilly et al European Patent Application Publication No. 125,023; Better et al., Science 240: 1041-1043 (1988); Liu et al., Proc. Natl. Acad. Sci. USA 84: 3439-3443 (1987); Liu et al., J. Immunol 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter, U.S. Pat. No. 5,225,539; Jones et al., Nature 321: 552-525 (1986); Verhoeyan et al., Science 239: 1534; and Beidler et al., J. Immunol. 141: 4053-4060 (1988).

Transgenic mice that are incapable of expressing endogenous immunoglobulin heavy and light chains genes, but that can express human heavy and light chain genes, may be used to produce human antibodies for use in the present teachings. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Life Technologies Corp. (Carlsbad, Calif.), Abgenix, Inc. (Fremont, Calif), and Medarex, Inc. (Princeton, N.J.), can be engaged to provide human antibodies directed against a selected antigen using technology similar to that described above. Human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

Oligosaccharides are attached to antibody molecules, such as IgG, at asparagine residues on the Fc portion of the antibody. At the amino acid, there are two GlcNAc sugars attached to each other by a beta (1-4) linkage. Enzymes such as endoglycosidases cleave this linkage, so that one GlcNAc residue remains attached to the asparagine on the IgG, while the other GlcNAc remains attached to the rest of the oligosaccharide. The GlcNAc attached to the oligosaccharide contains a reactive reducing-end, which can be selectively modified without altering the other sugar residues.

The enzyme galactosyl transferase normally transfers a galactose from UDP-galactose to a terminal GlcNAc residue. Khidekel et al (J. Am. Chem. Soc. 125:16162-16163 (2003); Hsieh-Wilson, L., et al., U.S. Patent Publication No. 2005/0130235) used a mutant galactosyl transferase, a Y289L mutant, to transfer an acetone-containing galactose substrate to a GlcNAc residue. An azide-containing galactose substrate (e.g., UDP-GalNAz) may be synthesized for transfer to the GlcNAc site by the mutant galactosyl transferase.

Unnatural sugar substrates may be synthesized that incorporate reactive chemical handles that may be used for click chemistry. The azide/alkyne cycloaddition reaction can be used to introduce affinity probes (biotin), dyes, polymers (e.g., poly(ethylene glycol) or polydextran) or other monosaccharides (e.g., glucose, galactose, fucose, O-GlcNAc, mannose-derived saccharides bearing the appropriate chemical handle). In certain embodiments, these handles include, for example, azide, triarylphosphine, activated alkyne, cyclooctyne or alkyne residues. The chemical handle also can be an azido group capable of reacting in a Staudinger reaction (see, for example, Saxon, E., et al., J. Am. Chem. Soc., 124(50): 14893-14902 (2002)). The phosphine can have a neighboring acyl group such as an ester, thioester or N-acyl imidazole (i.e. a phosphinoester, phosphinothioester, phosphinoimidazole) to trap the aza-ylide intermediate and form a stable amide bond upon hydrolysis. The phosphine can also be typically a di- or triarylphosphine to stabilize the phosphine.

Various labels or tags may be linked or conjugated to the glycoprotein using the methods disclosed herein. The labels or tags may also, for example, be detectable labels used, for example, for diagnostic or research purposes. Examples of such labels or tags include, but are not limited to fluorescent dyes, such as, for example, fluorescein (FITC), Oregon Green 488 dye, Marina Blue dye, Pacific Blue dye, and Texas Red-X dye, Alexa Fluor dyes (Life Technologies Corp., Carlsbad, CA); compounds containing radioisotopes; phycobiliproteins, such as, for example, R-phycoerythrin (R-PE, allophycocyanin (AP); and particles, such as, for example, Qdots, gold, ferrofluids, dextrans and microspheres.

The reporter molecules disclosed herein include any directly or indirectly detectable reporter molecule known by one skilled in the art that can be attached to a modified glycoprotein disclosed herein. Reporter molecules include, without limitation, a chromophore, a fluorophore, a fluorescent protein, a phosphorescent dye, a tandem dye, a particle, and a radioisotope. Preferred reporter molecules include fluorophores, fluorescent proteins and radioisotopes.

A fluorophore as described herein is any chemical moiety that exhibits an absorption maximum beyond 280 nm, and when covalently attached to a labeling reagent retains its spectral properties. Fluorophores used herein include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Ser. Nos. 09/968,401 and 09/969,853), a carbocyanine (including any corresponding compounds in U.S. patent application Ser. Nos. 09/557,275; 09/969,853 and 09/968,401; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; PCT International Publication Nos. WO 02/26891, WO 97/40104, WO 99/51702, WO 01/21624; and European Patent Application Publication No. 1 065 250 Al), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. ptent application Ser. No. 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045), or a rhodamine (including any corresponding compounds in U.S. Pat. Nos. 5,798,276; 5,846,737; U.S. patent application Ser. No. 09/129,015). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Preferred fluorophores include xanthene (rhodol, rhodamine, fluorescein and derivatives thereof) coumarin, cyanine, pyrene, oxazine and borapolyazaindacene. Most preferred are sulfonated xanthenes, fluorinated xanthenes, sulfonated coumarins, fluorinated coumarins and sulfonated cyanines. The choice of the fluorophore attached to the labeling molecule will determine the absorption and fluorescence emission properties of the labeling molecule and the labeled glycoprotein or labeled antibody. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

Suitable detectable labels include, for example, fluoresceins (e.g., 5-carboxy-2,7-dichlorofluorescein; 5-Carboxyfluorescein (5-FAM); 5-HAT (Hydroxy Tryptamine); 6-HAT; 6-JOE; 6-carboxyfluorescein (6-FAM); FITC); Alexa Fluor (AF) fluorophores (e.g., 350, 405, 430, 488, 500, 514, 532, 546, 555, 568, 594, 610, 633, 635, 647, 660, 680, 700, 750); BODIPY° fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), coumarins (e.g., 7-amino-4-methylcoumarin, AMC, AMCA, AMCA-S, AMCA-X, ABQ, CPM methylcoumarin, coumarin phalloidin, hydroxycoumarin, CMFDA, methoxycoumarin), calcein, calcein AM, calcein blue, calcium dyes (e.g., calcium crimson, calcium green, calcium orange, calcofluor white), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/tetramethylrhodamine, IAEDANS/fluorescein, EDANS/dabcyl, fluorescein/fluorescein, BODIPY° FL/BODIPY® FL, Fluorescein/QSY7 and QSY9), LysoTracker® and LysoSensor™ (e.g., LysoTracker® Blue DND-22, LysoTracker® Blue-White DPX, LysoTracker® Yellow HCK-123, LysoTracker® Green DND-26, LysoTracker® Red DND-99, LysoSensor™ Blue DND-167, LysoSensor™ Green DND-189, LysoSensor™ Green DND-153, LysoSensor™ Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, 5-ROX (carboxy-X-rhodamine), Sulphorhodamine B can C, Sulphorhodamine G Extra, Tetramethylrhodamine (TRITC), WT), Texas Red, Texas Red-X, VIC and other labels described in, e.g., US Pub. No. 2009/0197254), among others as would be known to those of skill in the art. Other detectable labels can also be used (see, e.g., U.S. Patent Application Publication No. 2009/0197254), as would be known to those of skill in the art.

In one aspect, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp). In certain embodiments, the fluorophores are near-IR (NIR) dyes (NIR=700-900 nm).

Many of fluorophores can also function as chromophores and thus the described fluorophores are also preferred chromophores.

Fluorescent proteins also find use as labels for the labeling molecules described herein. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorophore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. patent application Ser. Nos. 09/968/401 and 09/969/853; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor.

### Separation and Detection

Another aspect provided herein are methods directed to detecting modified glycoproteins after the modified glycoproteins have been labeled, using the methods described herein, and separated using, for example, chromatographic methods or electrophoresis methods such as, but not limited to, thin layer or column chromatography (including, for example, size exclusion, ion exchange, or affinity chromatography) or isoelectric focusing, gel electrophoresis, capillary electrophoresis, capillary gel electrophoresis, and slab gel electrophoresis. Gel electrophoresis can be denaturing or nondenaturing gel electrophoresis, and can include denaturing gel electrophoresis followed by nondenaturing gel electrophoresis (e.g., “2D” gels).

The modified glycoproteins that can be labeled, separated and detected using the methods described herein include, but are not limited to, antibodies and Fc-fusion proteins. In certain embodiments, such glycoproteins have been modified using the methods described herein.

In other embodiments, the separation methods used in such separation and detection methods can be any separation methods used for glycoproteins, such as, for example, chromatography, capture to solid supports, and electrophoresis. In certain embodiments, gel electrophoresis is used to separate glycoproteins, such as but not limited to antibodies. Gel electrophoresis is well known in the art, and in the context of the present disclosure can be denaturing or nondenaturing gel electrophoresis and can be 1D or 2D gel electrophoresis.

In certain embodiments of such separation and detection methods, gel electrophoresis is used to separate antibodies and the separated antibodies are detected in the gel by the attached labels. By way of example only, antibodies that have incorporated azido sugars can be labeled in a solution reaction with a terminal alkyne-containing fluorophore, and the antibodies can be optionally further purified from the reaction mixture and electrophoresed on a 1D or 2D gel. The antibodies can be visualized in the gel using light of the appropriate wavelength to stimulate the fluorophore label.

Gel electrophoresis can use any feasible buffer system described herein including, but not limited to, Tris-acetate, Tris-borate, Tris-glycine, BisTris and Bistris-Tricine. In certain embodiments, the electrophoresis gel used in the methods described herein comprise acrylamide, including by way for example only, acrylamide at a concentration from about 2.5% to about 30%, or from about 5% to about 20%. In certain embodiments, such polyacrylamide electrophoresis gels comprise 1% to 10% crosslinker, including but not limited to, bisacrylamide. In certain embodiments, the electrophoresis gel used in the methods described herein comprises agarose, including by way for example only, agarose at concentration from about 0.1% to about 5%, or from about 0.5% to about 4%, or from about 1% to about 3%. In certain embodiments, the electrophoresis gel used in the methods described herein comprises acrylamide and agarose, including by way for example only, electrophoresis gels comprising from about 2.5% to about 30% acrylamide and from about 0.1% to about 5% agarose, or from about 5% to about 20% acrylamide and from about 0.2% to about 2.5% agarose. In certain embodiments, such polyacrylamide/agarose electrophoresis gels comprise 1% to 10% crosslinker, including but not limited to, bisacrylamide. In certain embodiments, the gels used to separate glycoproteins can be gradient gels.

The methods described herein can be used to detect modified glycoproteins for “in-gel” detection using slab gel electrophoresis or capillary gel electrophoresis. In certain embodiments such modified glycoproteins are antibodies or Fc-fusion proteins.

In-gel fluorescence detection allows for quantitative differential analysis of protein glycosylation between different biological samples and is amenable to multiplexing with other protein gel stains. In certain embodiments of the methods described herein, utilizing fluorescent- and/or UV-excitable alkyne containing probes, or fluorescent- and/or UV-excitable azide containing probes, allow for the multiplexed detection of glycoproteins, phosphoproteins, and total proteins in the same 1-D or 2-D gels.

The compounds and compositions described herein may, at any time before, after or during an assay, be illuminated with a wavelength of light that results in a detectable optical response, and observed with a means for detecting the optical response. In certain embodiments, such illumination can be by a violet or visible wavelength emission lamp, an arc lamp, a laser, or even sunlight or ordinary room light, wherein the wavelength of such sources overlap the absorption spectrum of a fluorophore or chromophore of the compounds or compositions described herein. In certain embodiments, such illumination can be by a violet or visible wavelength emission lamp, an arc lamp, a laser, or even sunlight or ordinary room light, wherein the fluorescent compounds, including those bound to the complementary specific binding pair member, display intense visible absorption as well as fluorescence emission.

In certain embodiments, the sources used for illuminating the fluorophore or chromophore of the compounds or compositions described herein include, but are not limited to, hand-held ultraviolet lamps, mercury arc lamps, xenon lamps, argon lasers, laser diodes, blue laser diodes, and YAG lasers. These illumination sources are optionally integrated into laser scanners, flow cytometer, fluorescence microplate readers, standard or mini fluorometers, or chromatographic detectors. The fluorescence emission of such fluorophores is optionally detected by visual inspection, or by use of any of the following devices: CCD cameras, video cameras, photographic film, laser scanning devices, fluorometers, photodiodes, photodiode arrays, quantum counters, epifluorescence microscopes, scanning microscopes, flow cytometers, fluorescence microplate readers, or by means for amplifying the signal such as photomultiplier tubes. Where the sample is examined using a flow cytometer, a fluorescence microscope or a fluorometer, the instrument is optionally used to distinguish and discriminate between the fluorescent compounds described herein and a second fluorophore with detectably different optical properties, typically by distinguishing the fluorescence response of the fluorescent compounds described herein from that of the second fluorophore. Where a sample is examined using a flow cytometer, examination of the sample optionally includes isolation of particles within the sample based on the fluorescence response by using a sorting device.

In certain embodiments, fluorescence is optionally quenched using either physical or chemical quenching agents.

In certain embodiments, the labeled glycoproteins may be used to perform diagnostic imaging. The imaging technique may include whole body imaging for diagnostic purposes or local imaging at specific sites, such as but not limited to sites of tumor growth, in a quantitative manner to assess the progression of disease or host response to a treatment regimen. The imaging may be accomplished in vitro or in vivo by any suitable method known in the art. For example, and without wishing to be limiting, the diagnostic imaging technique may include immunohistochemistry, immunofluorescence staining, or a non-invasive (molecular) diagnostic imaging technology including, but not limited to: optical imaging; positron emission tomography (PET), wherein the detectable agent is an isotopes such as ¹¹C, ¹³N, ¹⁵O, ¹⁸F, ⁶⁴Cu, ⁶²Cu, ¹²⁴I, ⁷⁶Br, ⁸²Rb and ⁶⁸Ga; or single photon emission computed tomography (SPECT), wherein the detectable agent is a radiotracer such as ^{90m}Tc, ¹¹¹In, ¹²³I, ²⁰¹Tl, ¹³³Xe, depending on the specific application.

### Samples and Sample Preparation

The end user will determine the choice of the sample and the way in which the sample is prepared. Samples that can be used with the methods and compositions described herein include, but are not limited to, any biological derived material or aqueous solution that contains a cell-associated antigen or analyte. In certain embodiments, a sample also includes material in which a modified glycoprotein has been added. The sample that can be used with the methods and compositions described herein can be a biological fluid including, but not limited to, whole blood, plasma, serum, nasal secretions, sputum, saliva, urine, sweat, transdermal exudates, cerebrospinal fluid, or the like. In other embodiments, the samples are biological fluids that include tissue and cell culture medium wherein modified biomolecule of interest has been secreted into the medium. Cells used in such cultures include, but are not limited to, prokaryotic cells and eukaryotic cells that include primary cultures and immortalized cell lines. Such eukaryotic cells include, without limitation, ovary cells, epithelial cells, circulating immune cells, _{R} cells, hepatocytes, and neurons. In certain embodiments, the sample may be whole organs, tissue or cells from an animal, including but not limited to, muscle, eye, skin, gonads, lymph nodes, heart, brain, lung, liver, kidney, spleen, thymus, pancreas, solid tumors, macrophages, mammary glands, mesothelium, and the like. In certain embodiments, the sample may be a subject, such as a mammal.

Various buffers can be used in the methods described herein, including inorganic and organic buffers. In certain embodiments the organic buffer is a zwitterionic buffer. By way of example only, buffers that can be used in the methods described herein include phosphate buffered saline (PBS), phosphate, succinate, citrate, borate, maleate, cacodylate, N-(2-Acetamido)iminodiacetic acid (ADA), 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxypropanesulfonic acid (MOPSO), N,N-bis-(hydroxyethyl)-2-aminoethanesulfonic acid (BES), 3-(N-morpholino)-propanesulfonic acid (MOPS), N-tris-(hydroxymethyl)-2-ethanesulfonic acid (TES), N-2-hydroxyethyl-piperazine-N-2-ethanesulfonic acid (HEPES), 3-(N-tris-(hydroxymethyl) methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)piperazine-N′-(2-hydroxypropanesulfonic acid) (HEPPSO), 4-(2-Hydroxyethyl)-1-piperazinepropanesulfonic acid (EPPS), N-[Tris(hydroxymethyl)methyl]glycine (Tricine), N,N-Bis(2-hydroxyethyl)glycine (Bicine), (2-Hydroxy-1,1-bis(hydroxymethyl)ethyl)amino]-1-propanesulfonic acid (TAPS), N-(1,1-Dimethyl-2-hydroxyethyl)-3-amino-2-hydroxypropanesulfonic acid (AMPSO), tris (hydroxy methyl) amino-methane (Tris), Tris-Acetate-EDTA (TAE), glycine, bis[2-hydroxyethyl]iminotris[hydroxymethyl]methane (BisTris), or combinations thereof. In certain embodiments, wherein such buffers are used in gel electrophoresis separations the buffer can also include ethylene diamine tetraacetic acid (EDTA).

The concentration of such buffers used in the methods described herein is from about 0.1 mM to 1 M. In certain embodiments the concentration is between 10 mM to about 1 M. In certain embodiments the concentration is between about 20 mM and about 500 mM, and in other embodiments the concentration is between about 50 mM and about 300 mM. In certain embodiments, the buffer concentration is from about 0.1 mM to about 50 mM, while in other embodiments the buffer concentration if from about 0.5 mM to about 20 mM.

In certain embodiments, buffers used in the methods described herein have a pH between 5 and 9 at ambient temperature. In certain embodiments the buffer has a pH between 6 and 8.5 at ambient temperature. In certain embodiments the buffer has a pH between 6 and 8 at ambient temperature. In certain embodiments the buffer has a pH between 6 and 7 at ambient temperature. In certain embodiments the buffer has a pH between 5 and 9 at 25° C. In certain embodiments the buffer has a pH between 6 and 8.5 at 25° C. In certain embodiments the buffer has a pH between 6 and 8 at 25° C. In certain embodiments the buffer has a pH between 6 and 7 at 25° C.

In certain embodiments, the samples used in the methods described herein contain a non-ionic detergent. Non-limiting examples of such non-ionic detergents added to the samples used in the methods described herein are polyoxyalkylene diols, ethers of fatty alcohols including alcohol ethoxylates (Neodol from Shell Chemical Company and

Tergitol from Union Carbide Corporation), alkyl phenol ethoxylates (Igepal surfactants from General Aniline and Film Corporation), ethylene oxide/propylene oxide block copolymers (PLURONIC™ Series from BASF Wyandotte Corporation), polyoxyethylene ester of a fatty acids (Stearox CD from Monsanto Company), alkyl phenol surfactants (Triton series, including Triton X-100 from Rohm and Haas Company), polyoxyethylene mercaptan analogs of alcohol ethoxylates (Nonic 218 and Stearox SK from Monsanto Company), polyoxyethylene adducts of alkyl amines (Ethoduomeen and Ethomeen surfactants from Armak Company), polyoxyethylene alkyl amides, sorbitan esters (such as sorbitan monolaurate) and alcohol phenol ethoxylate (Surfonic from Jefferson Chemical Company, Inc.). Non-limiting examples of sorbitan esters include polyoxyethylene(20) sorbitan monolaurate (TWEEN20), polyoxyethylene(20) sorbitan monopalmitate (TWEEN40), polyoxyethylene(20) sorbitan monostearate (TWEEN60) and polyoxyethylene(20) sorbitan monooleate (TWEEN 80). In certain embodiments, the concentration of such non-ionic detergents added to a sample is from 0.01 to 0.5%. In other embodiments the concentration is from about 0.01 to 0.4 vol. %. In other embodiments the concentration is from about 0.01 to 0.3 vol. %. In other embodiments the concentration is from about 0.01 to 0.2 vol. %. In other embodiments the concentration is from about 0.01 to 0.1 vol. %.

### Compositions:

In another aspect, compositions are provided for use in the methods provided herein. In certain embodiments, the compositions comprise a labeling molecule that comprises a metal ion chelator and a reactive group. In certain embodiments, the labeling molecule further comprises a fluorophore. In certain embodiments, the labeling molecule comprises a metal ion chelator, a reactive group, and a fluorophore. In certain embodiments, the compositions comprise a labeling molecule that comprises a reactive group and a fluorophore. In certain embodiments, the compositions comprise a tyrosine, a fluorophore, and a reactive group. In certain embodiments, the compositions comprise a labeling molecule having Formula (I):

FLUOROPHORE-REACTIVE GROUP-METAL ION CHELATOR (I)

wherein,

FLUOROPHORE is a coumarin, a cyanine, a benzofuran, a quinolone, a quinazoline, an indole, a benzazole, a borapolyazaindacine, or a xanthene;

REACTIVE GROUP comprises a terminal triarylphosphine, an alkyne, a terminal alkyne, an activated alkyne group, an azide, a ketone, a hydrazide, a semicarbazide, a thiocarbonylhydrazide, a carbonylhydrazide, a thiocarbonylhydrazide, a sulfonylhydrazide, a carbazide, a thiocarbazide, or an aminooxy group, a Diels-Alder diene, a Diels-Alder dienophile; and

METAL ION CHELATOR is a1,4,8,11-tetraazabicyclo[6.6.2]hexadecane-4,11-diyl)diacetic acid (CB-TE2A); desferrioxamine; diethylenetriaminepentaacetic acid (DTPA); 1,4,7,10-tetraazacyclotetradecane-1,4,7,10-tetraacetic acid (DOTA); ethylenediaminetetraacetic acid (EDTA); ethylene glycolbis(2-aminoethyl)-N,N,N′,N′-tetraacetic acid (EGTA); 1,4,8,11-tetraazacyclotetradecane-1,4,8,11-tetraacetic acid (TETA); ethylenebis-(2-4 hydroxy-phenylglycine) (EHPG); 5-Cl-EHPG; 5Br-EHPG; 5-Me-EHPG; 5t-Bu-EHPG; 5-sec-Bu-EHPG; benzodiethylenetriamine pentaacetic acid (benzo-DTPA); dibenzo-DTPA; phenyl-DTPA, diphenyl-DTPA; benzyl-DTPA; dibenzyl DTPA; bis-2 (hydroxybenzyl)-ethylene-diaminediacetic acid (HBED) and derivatives thereof; Ac-DOTA; benzo-DOTA; dibenzo-DOTA; NOTA (1,4,7-triazacyclononane N,N′,N”-triacetic acid); benzo-NOTA; benzo-TETA, benzo-DOTMA, where DOTMA is 1,4,7,10-tetraazacyclotetradecane-1,4,7,10-tetra(methyl tetraacetic acid), benzo-TETMA, where TETMA is 1,4,8,11-tetraazacyclotetradecane-1,4,8,11-(methyl tetraacetic acid); derivatives of 1,3-propylenediaminetetraacetic acid (PDTA); triethylenetetraaminehexaacetic acid (TTHA); derivatives of 1,5,10-N,N′,N″-tris(2,3-dihydroxybenzoyl)-tricatecholate (LICAM); and 1,3,5-N,N′,N″-tris(2,3-dihydroxybenzoyl)aminomethylbenzene (MECAM).

In certain embodiments, the composition comprises a labeling molecule of Formula (I), wherein the fluorophore is selected from a xanthene, a cyanine, a borapolyazaindacine, and a coumarin; the reactive group is an activated alkyne group; and the metal ion chelator is selected from DFO, NOTA and DOTA.

In certain embodiments, the composition comprises a labeling molecule of Formula (I), wherein the fluorophore is selected from a xanthene, a cyanine, a borapolyazaindacine, and a coumarin; the reactive group is a cyclooctyne; and the metal ion chelator is selected from DFO, NOTA and DOTA.

In certain embodiments, the composition comprises a labeling molecule of Formula (I), wherein fluorophore is selected from a xanthene, a cyanine, a borapolyazaindacine, and a coumarin; the reactive group is a DIBO; and the metal ion chelator is selected from DFO, NOTA and DOTA.

### Kits:

In another aspect, kits are provided for use in the methods provided herein. In certain embodiments, kits are provided for labeling a glycoprotein that include a modified sugar comprising a chemical handle, and a labeling molecule comprising a metal ion chelator group and a reactive group. In certain embodiments, the kits further comprise instructions for using the components in any of the methods as described herein. In certain embodiments, kits are provided for dual-labeling a glycoprotein that include a modified sugar comprising a chemical handle, and a labeling molecule comprising a metal ion chelator group, a reactive group and a fluorophore. In certain embodiments, the kits further include instructions for using the components in any of the methods as described herein. In certain embodiments, kits are provided for dual-labeling a glycoprotein that include a modified sugar comprising a chemical handle, a first labeling molecule comprising a metal ion chelator and a reactive group, and a second labeling molecule comprising a fluorophore and a reactive group. In certain embodiments, the kits further include instructions for using the components in any of the methods as described herein. In certain embodiments, kits are provided for labeling glycoproteins comprising a modified sugar comprising a chemical handle, and a labeling molecule comprising a tyrosine group, a reactive group, and a fluorophore. In certain embodiments, the kits further include instructions for using the components in any of the methods described herein.

In certain embodiments, kits are provided for detecting a cell-associated antigen that include a modified sugar comprising a chemical handle, and a labeling molecule comprising a metal ion chelator group and a reactive group. In certain embodiments, the kits further include instructions for using the components in any of the methods as described herein. In certain embodiments, kits are provided for detecting a cell-associated antigen that include a modified sugar comprising a chemical handle, and a labeling molecule comprising a metal ion chelator group, a reactive group, and a fluorophore. In certain embodiments, the kits further include instructions for using the components in any of the methods as described herein. In certain embodiments, kits are provided for detecting a cell-associated antigen that include a modified sugar comprising a chemical handle, a first labeling molecule comprising a metal ion chelator and a reactive group, and a second labeling molecule comprising a fluorophore and a reactive group. In certain embodiments, the kits further include instructions for using the components in any of the methods as described herein. In certain embodiments, kits are provided for detecting a cell-associated antigen comprising a modified sugar comprising a chemical handle, and a labeling molecule comprising a tyrosine group, a reactive group, and a fluorophore. In certain embodiments, the kits further include instructions for using the components in any of the methods described herein.

In certain embodiments, the kits may further include one or more of the following: an endoglycosidase, a sialidase, a β-galactosidase, a galactosyl transferase, a mutant galactosyl transferase, a Y289L mutant galactosyl transferase, a glycoprotein, an antibody, an Fc-fusion protein, and a radioactive metal ion. In certain embodiments, the kits may further include one or more of the following: one or more buffers, detergents and/or solvents.

The kits disclosed herein may also comprise one or more of the components in any number of separate containers, packets, tubes, vials, microtiter plates and the like, or the components may be combined in various combinations in such containers. For the kits disclosed herein, for example, the modified sugar comprising the chemical handle may be provided in a separate container than the labeling molecules.

The kits disclosed herein may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

A detailed description of the present teachings having been provided above, the following examples are given for the purpose of illustrating the present teachings and shall not be construed as being a limitation on the scope of the invention or claims.

The following examples are intended to illustrate but not limit the present disclosure.

### EXAMPLES

### Example 1

### Site-Specific Radiolabeling of J591 Antibodies With a DIBO-DFO Labeling Molecule

Reagents and General Procedures:

All chemicals, unless otherwise noted, were acquired from Sigma-Aldrich (St. Louis, Mo.) and were used as received without further purification. All water employed was ultra-pure (>18.2 MΩcm⁻¹ at 25° C.), all DMSO was of molecular biology grade (>99.9%), and all other solvents were of the highest grade commercially available. Deimmunized J591 was obtained through Memorial Sloan Kettering Cancer Center (MSKCC) Clinical Research Department/Weill Cornell Medical College. p-SCN-DFO was obtained from Macrocyclics, Inc. (Dallas, Tex.). All instruments were calibrated and maintained in accordance with standard quality-control procedures. UV-Vis measurements were taken on a Thermo Scientific NanoDrop 2000 Spectrophotometer.

⁸⁹Zr was produced at Memorial Sloan-Kettering Cancer Center on an EBCO TR19/9 variable-beam energy cyclotron (Ebco Industries, Inc., British Columbia, Canada) via the ⁸⁹Y(p,n)⁸⁹Zr reaction and purified in accordance with previously reported methods to yield ⁸⁹Zr with a specific activity of 5.3-13.4 mCi/μg (195 -497 MBq/μg) (Holland et al., Nucl. Med. Biol. 36:729-739 (2009)). Activity measurements were made using a Capintec CRC-15R Dose Calibrator (Capintec, Ramsey, N.J.). For accurate quantification of activities, experimental samples were counted for 1 min on a calibrated Perkin Elmer (Waltham, Mass.) Automatic Wizard Gamma Counter (Hang et al., Proc. Natl. Acad. Sci. USA 100:14846-14851 (2003)). Labeling of antibodies with ⁸⁹Zr was monitored using silica-gel impregnated glass-fiber instant thin-layer chromatography paper (Pall Corp., East Hills, N.Y.) and analyzed on a Bioscan AR-2000 radio-TLC plate reader using Winscan Radio-TLC software (Bioscan Inc., Washington, D.C.). All experiments performed on laboratory animals were performed according to a protocol approved by the Memorial Sloan-Kettering Institutional Animal Care and Use Committee (protocol 08-07-013).

Cell Culture:

Human prostate cancer cell line LNCaP was obtained from the American Tissue Culture Collection (ATCC, Manassas, Va., USA) and maintained by weekly serial passage in a 5% CO₂ (g) atmosphere at 37° C. Cells were harvested by using a formulation of 0.25% trypsin and 0.53 mM EDTA in Hank's buffered salt solution without calcium or magnesium. LNCaP cells were grown in RPMI 1640 medium supplemented with 10% fetal calf serum, 2 mM L-glutamine, 1 mM sodium pyruvate, 4.5 g/L glucose, 1.5 g/L sodium bicarbonate and 100 U/mL of penicillin and streptomycin.

Xenograft Models:

All experiments were performed under an Institutional Animal Care and Use Committee-approved protocol, and the experiments followed Institutional guidelines for the proper and humane use of animals in research. Six-eight week old athymic nude male (Hsd: Athymic Nude-nu) mice were obtained from Harlan Laboratories (Indianapolis, Ind.). Animals were housed in ventilated cages, were given food and water ad libitum, and were allowed to acclimatize for approximately 1 week prior to inoculation. LNCaP tumors were induced on the right shoulder by a subcutaneous injection of 5.0×10⁶ cells in a 200 μL cell suspension of a 1:1 mixture of fresh media:BD matrigel (BD Biosciences, Bedford, Mass.). The xenografts reached ideal size for imaging and biodistribution (˜100-150 mm³) in approximately 4 weeks.

Synthesis of N-azidoacetylgalactosamine (UDP-GalNAz):

UDP-GalNAz was synthesized in accordance with previously reported methods (Hang, et al., Proc. Natl. Acad. Sci. USA 100:14846-14851 (2003)).

Synthesis of DIBO-DFO:

To a suspension of 1-(4-isothiocyanatophenyl)-3-[6,17-dihydroxy-7,10,18,21-tetraoxo-27-(N-acetylhydroxylamino)-6,11,17,22-tetraheptaeocpsome]thiourea (p-NCS-Bn-Deferroxamine (p-NCS-DFO), 22 mg, 27 μmol) and N4242-(2-aminoethoxy)ethoxy]ethyl]2-(11, 12-didehydro-5,6-dihydrodobenzo[a,e]cycloocten-5-yl)oxy]acetamide (DIBO amine, 20 mg, 54 μmol) (Ning, et al., Angew. Chem. Int. Ed. 47:2253-2255 (2008)) in 1.5 mL of anhydrous DMF was added triethylamine (75 μL, 0.54 mmol) and the mixture was stirred at room temperature for 48 hours. The resulting reaction mixture, which became a homogeneous solution, was added into 25 mL of ethyl acetate slowly over a 2 minute period while stirring vigorously at room temperature. The resulting precipitate was collected by filtration to give the desired product (DIBO-DFO, 24 mg, 80% yield) as an off-white solid. TLC (silica gel, 15% H₂O in CH₃CN): R_{f}=0.59.

Modification of J591 With DFO-DIBO/GalNAz:

Glycans Modification: J591 (1 mg, 8 mg/mL) underwent a buffer exchange into pre-treatment buffer (50 mM Na-phosphate, pH 6.0) using a micro-spin column prepared with P30 resin (Bio-Rad 732-6008, 1.5 bed volume). The column was first equilibrated in 50 mM Na-phosphate, pH 6.0, and then spun for 3 minutes at 850×g, 125 μL J591 antibody was added, and spun down for 5 minutes at 850×g. The resultant antibody solution was supplemented with 40 μL of β-1,4-galactosidase (from S. pneumonia (2 mU/μL)) and placed in an incubator at 37° C. overnight.

GalNAz Labeling: A buffer exchange of the sample into TBS reaction buffer (20 mM Tris HCl, 0.9% NaCl, pH 7.4) was performed using a micro-spin column prepared with P30 resin. After the buffer exchange, the antibody (600 μg in 300 μL TBS buffer) was combined with UDP-GalNAz (40 ∥L of a 40 mM solution in H₂O), MnCl₂ (150 μL of a 0.1 M solution), and GalT (Y289L) (1000 μL of 0.29 mg/mL in 50 mM Tris, 5 mM EDTA (pH 8)). The final solution contained concentrations of 0.4 mg/mL antibody, 10 mM MnCl₂, 1 mM UDP-GalNAz, and 0.2 mg/mL GalT (Y289L). The resultant solution was incubated overnight at 30° C.

DIBO-DFO Labeling: The solution from the GalNAz labeling step was purified using six micro-spin columns prepared with P30 resin and TBS buffer (each micro-spin column received 250 μL of the GalNAz labeling solution). After centrifugation, the filtrates were combined to yield 1500 μL of antibody solution. Subsequently, 200 μL of DIBO-DFO solution (1.74 mg in 750 μL DMSO, 2 mM stock) was added to the combined filtrates, and this tube was incubated at 25° C. overnight.

Purification: After DIBO-DFO labeling, the completed antibody was purified via size exclusion chromatography (PD10 column, GE healthcare) and concentrated using centrifugal filter units with a 50,000 molecular weight cut off (AMICON Ultra 4 Centrifugal Filtration Units, Millipore Corp., Billerica, MA) and phosphate buffered saline (PBS, pH 7.4).

Modification of J591 With DFO-NCS:

J591 (2-3 mg) was dissolved in 1 mL of phosphate buffered saline (pH 7.4), and the pH of the solution was adjusted to 8.8-9.0 with NaHCO₃ (0.1 M). To this solution was added an appropriate volume of NCS-DFO in DMSO (5-10 mg/mL) to yield a chelator:antibody reaction stoichiometry of 6:1. The resultant solution was incubated with gentle shaking for 30 min at 37° C. After 30 min, the modified antibody was purified using centrifugal filter units with a 50,000 molecular weight cut off (AMICON Ultra 4 Centrifugal Filtration Units, Millipore Corp, Billerica, MA) and phosphate buffered saline (PBS, pH 7.4) (Vosjan, et al., Nat. Prot. 5:739-743 (2010)).

SDS-PAGE Confirmation of Modification Sites on Heavy Chain N-Linked Glycans:

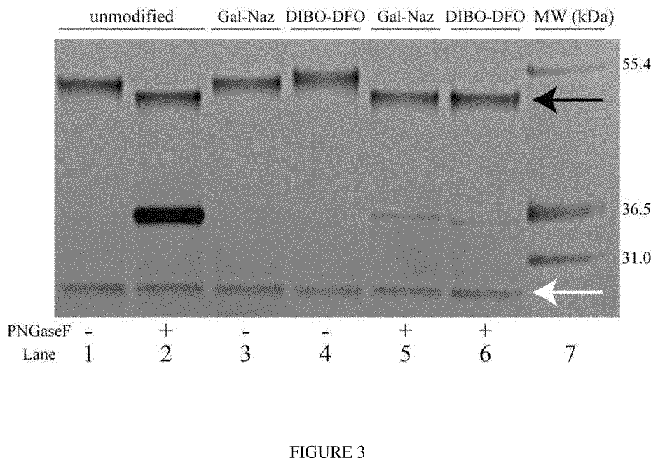

The N-glycans of J591 were GalNAz-tagged at the terminal GlcNAc residues with UDP-GalNAz, using the β-galactosyltransferase mutant Y289L (FIG. 3, lanes 3-6). The azide groups were then click reacted with DIBO-DFO (FIG. 3, lanes 4, 6) or left unmodified (FIG. 3, lanes 3, 5). The N-glycans on the Fc of the heavy chain were then retained (FIG. 3, lanes 3, 4) or removed from their asparagines residue attachment points via PNGase F treatment (FIG. 3, lanes 5, 6). In addition, control, unmodified JH591 was also included either treated (FIG. 3, lane 2) or untreated (FIG. 3, lane 1) with PNGase F. MARK12 Unstained Standard (Life Technologies, Carlsbad, Calif.) was used as the molecular weight standard (FIG. 3, lane 7).

SDS-PAGE was performed on NuPAGE 4-12% in MOPS in running buffer. For gel analysis, antibodies were applied on NuPAGE 4-12% Bis-Tris gels and run in MOPS buffer. 200 ng antibody was applied per lane. After staining with SYPRO Ruby Protein Stain, the gels were imaged with FUJI FLA9000 with an excitation of 473 nm and a 575LP filter.

PNGase F Treatment of Antibody J591:

J591 antibody construct (1 μg) in 10 μl TBS was denatured with 0.5% SDS and 40 mM DTT by adding 17 μL H₂O and 3 μL 10× Glycoprotein Denaturation Buffer (New England Biolabs, Ipswich, Mass.) and incubation at 90° C. for 10 min. For PNGase F treatment, 18 μL H2O, 6 μL 10% NP-40 and 6 μL 500 mM sodium phosphate, pH 7.5 (G7 reaction buffer from New England Biolabs) was added. Samples were split in half, and one aliquot was supplemented with 1 μL PNGase F (New England Biolabs) and incubated overnight at 37° C. 12 μL were loaded per lane on a SDS gel for analysis.

Radiolabeling of Antibody Constructs with ⁸⁹Zr:

For each antibody construct (0.4-0.5 mg) was added to 200 μL buffer (PBS, pH 7.4). [⁸⁹Zr]Zr-oxalate (2000-2500 μCi) in 1.0 M oxalic acid was adjusted to pH 7.2-8.5 with 1.0 M Na2CO₃. After evolution of CO₂ (g) stops, the ⁸⁹Zr solution was added to the antibody solution, and the resultant mixture was incubated at room temperature for 1 h. After 1 h, the reaction progress was assayed using radio-TLC with an eluent of 50 mM EDTA, pH 5, and the reaction was quenched with 50 μL of the same EDTA solution. The antibody construct was purified using size-exclusion chromatography (Sephadex G-25 M, PD-10 column, GE Healthcare; dead volume=2.5 mL, eluted with 500 mL fractions of PBS, pH 7.4) and concentrated, if necessary, with centrifugal filtration. The radiochemical purity of the crude and final radiolabeled bioconjugate was assayed by radio-ITLC. In the ITLC experiments, the antibody construct remains at the baseline, while ⁸⁹Zr⁴⁺ ions and [⁸⁹Zr]-EDTA elute with the solvent front.

Immunoreactivity:

The immunoreactivity of the ⁸⁹Zr-DFO-DIBO/GalNAz-J591 and ⁸⁹Zr-NCS-DFO-J591 bioconjugates was determined using specific radioactive cellular-binding assays following procedures derived from Lindmo, et al., J. Immunol. Meth. 72:77-89 (1984) and Lindmo, et al., Methods Enzymol. 121:678-691 (1986), both of which are herein incorporated by reference in their entirety). To this end, LNCaP cells were suspended in microcentrifuge tubes at concentrations of 5.0, 4.0, 3.0, 2.5, 2.0, 1.5, and 1.0×10⁶ cells/mL in 500 μL PBS (pH 7.4). Aliquots of either ⁸⁹Zr-DFO-DIBO/GalNAz-J591 and ⁸⁹Zr-NCS-DFO-J591 (50 μL of a stock solution of 10 μCi in 10 mL of ⅕ bovine serum albumin in PBS, pH 7.4) were added to each tube (n=4; final volume: 550 μL), and the samples were incubated on a mixer for 60 min at room temperature. The treated cells were then pelleted via centrifugation (3000 rpm for 5 min), resuspended, and washed twice with cold PBS before removing the supernatant and counting the activity associated with the cell pellet. The activity data were background-corrected and compared with the total number of counts in appropriate control samples. Immunoreactive fractions were determined by linear regression analysis of a plot of (total/bound) activity against (1/[normalized cell concentration]). No weighting was applied to the data, and data were obtained in triplicate.

Stability Measurements:

The stability of the ⁸⁹Zr-DFO-DIBO/GalNAz-J591 and ⁸⁹Zr-NCS-DFO-J591 bioconjugates with respect to radiochemical purity and loss of radioactivity from the antibody was investigtated in vitro by incubation of the antibodies in human serum for 7 days (⁸⁹Zr) both at room temperature and 37° C. The radiochemical purity of the antibodies was determined via radio-TLC with an eluent of 50 mM EDTA, pH 5.0. All experiments were performed in triplicate. Both final constructs, ⁸⁹Zr-DFO-DIBO/GalNAz-J591 and ⁸⁹Zr-NCS-DFO-J591, demonstrated >96% stability at 120 hrs.

Chelate Number:

The number of accessible DFO chelates conjugated to each antibody was measured by radiometric isotopic dilution assays following methods similar to those described by Anderson, et al., J. Nucl. Med. 33:1685-1691 (1992) and Holland, et al., Plos One 5 (2010), both of which are herein incorporated by reference in their entirety.

PET Imaging:

PET imaging experiments were conducted on a microPET Focus rodent scanner (Concorde Microsystems). Mice bearing subcutaneous LNCaP (right shoulder) xenografts (100-150 mm3) were administered ⁸⁹Zr-DFO-DIBO/GalNAz-J591 or ⁸⁹Zr-NCS-DFO-J591 (10.2-12.0 MBq (275-325 μCi) in 200 μL, 0.9% sterile saline) via intravenous tail vein injection (t=0). Approximately 5 minutes prior to the PET images, mice were anesthetized by inhalation of 2% isolurane (Baxter Healthcare, Deerfield, Ill.)/oxygen gas mixture. Pet data for each mouse were recorded via static scans at various time points between 24 and 120 h. A minimum of 20 million coincident events were recorded for each scan, which lasted between 10-45 min. An energy window of 350-700 keV and a coincidence timing window of 6 ns were used. Data were sorted into 2-dimensional histograms by Fourier re-binning, and transverse images were reconstructed by filtered back-projection (FBP) into a 128×128×63 (0.72×0.72×1.3 mm) matrix. the image data were normalized to correct for non-uniformity of response of the PET, dead-time count losses, positron branching ratio, and physical decay to the time of injection but no attenuation, scatter or partial-volume averaging correction was applied. The counting rates in the reconstructed images were converted to activity concentration (percentage injected dose (% ID) per gram of tissue) by use of a system calibration factor derived from the imaging of a mouse-sized water-equivalent phantom containing ⁸⁹Zr. Images were analyzed using ASIPro VM™ software (Concorde Microsystems).

Acute Biodistribution:

Acute in vivo biodistribution studies were performed in order to evaluate the uptake of both ⁸⁹Zr-DFO-DIBO/GalNAz-J591 and ⁸⁹Zr-NCS-DFO-J591 in mice bearing subcutaneous LNCaP (right shoulder) xenografts (100-150 mm3, 4 weeks post inoculation). Tumor-bearing mice were randomized before the study and were warmed gently with a heat lamp for 5 min before administration of the appropriate ⁸⁹Zr-antibody construct (0.55-0.75 MBq (15-20 μCi) in 200 μL 0.9% sterile saline, 4-6 μg) via intravenous tail vein injection (t=0). Animals (n=4 per group) were euthanized by CO₂ (g) asphyxiation at 24, 48, 72, 96 h (⁸⁹Zr). A blocking experiment was also employed at the 72 h experiment, in which animals were given the same radioactive dose but with the addition of 200 μg of cold, unlabeled J591. After asphyxiation, 13 tissues (including tumor) were removed, rinsed in water, dried in air for 5 min, weighed, and counted in a gamma counter calibrated for ⁸⁹Zr. Counts were converted into activity using a calibration curve generated from known standards. Count data were background- and decay-corrected to the time of injection, and the percent injected dose per gram (% ID/g) for each tissue sample was calculated by normalization to the total activity injected.

Table 2 shows the biodistribution data for ⁸⁹Zr-DFO-DIBO/GalNAz-J591 versus time in mice bearing subcutaneous LNCaP xenografts (n=4 for each time point). Mice were administered ⁸⁹Zr-DFO-DIBO/GalNAz-J591 (0.55-0.75 MBq [15-20 μCi] in 200 μL 0.9% sterile saline) via tail vein injection (t=0). A blocking experiment was performed for the 72 h time point via co-injection of 300 μg non-radiolabeled J591 with the radiolabeled construct.

TABLE 2

| | 24 h | 48 h | 72 h | 96 h | 72 h block |
|---|---|---|---|---|---|
| Blood | 11.1 ± 4.5 | 7.2 ± 4 | 6 ± 1.6 | 2.9 ± 2.5 | 10.1 ± 1.9 |
| Tumor | 14.4 ± 2.5 | 28.2 ± 6.8 | 56.3 ± 5.1 | 67.6 ± 5 | 28.5 ± 6.8 |
| Heart | 4.6 ± 0.7 | 3.2 ± 1.2 | 3.1 ± 0.7 | 2.4 ± 0.8 | 3.2 ± 0.7 |
| Lung | 5.4 ± 2.5 | 2.6 ± 0.3 | 5.4 ± 0.6 | 1.4 ± 0.2 | 5.4 ± 1 |
| Liver | 10.3 ± 6.6 | 6.2 ± 4.6 | 5.1 ± 1.6 | 3.1 ± 0.9 | 4.7 ± 0.9 |
| Spleen | 11.1 ± 3 | 7 ± 5.7 | 2.7 ± 0.9 | 2 ± 0.7 | 3.3 ± 1.1 |
| Stomach | 0.8 ± 0.2 | 0.8 ± 0.1 | 0.5 ± 0.3 | 0.7 ± 0.5 | 0.7 ± 0.2 |
| Large | 1 ± 0.4 | 1.2 ± 1.2 | 0.4 ± 0.1 | 0.5 ± 0.4 | 0.5 ± 0.1 |
| Intestine | | | | | |
| Small | 2.4 ± 1 | 1.7 ± 1.3 | 0.9 ± 0.2 | 1.3 ± 1 | 1.4 ± 0.6 |
| Intestine | | | | | |
| Kidney | 5 ± 3.5 | 2.4 ± 1.2 | 2.6 ± 1.3 | 2.5 ± 1.2 | 3.9 ± 1.4 |
| Muscle | 1.6 ± 0.5 | 2.3 ± 0.8 | 2 ± 1 | 1 ± 0.4 | 1 ± 0.1 |
| Bone | 11.1 ± 6.6 | 10.3 ± 2.1 | 8.7 ± 0.8 | 7.4 ± 0.9 | 3.4 ± 1.4 |
| Skin | 3.1 ± 2.9 | 3.4 ± 1.6 | 3.1 ± 0.7 | 2.3 ± 0.5 | 5 ± 0.8 |

Table 3 shows biodistribution data for ⁸⁹Zr-DFO-NCS-J591 versus time in mice bearing subcutaneous LNCaP xenografts (n=4 for each time point). Mice were administered ⁸⁹Zr-DFO-NCS-J591 (0.55-0.75 MBq [15-20 μCi] in 200 μL 0.9% sterile saline) via tail vein injection (t=0). A blocking experiment was performed for the 72 h time point via co-injection of 300 μg non-radiolabeled J591 with the radiolabeled construct.

TABLE 3

| | 24 h | 48 h | 72 h | 96 h | 72 h block |
|---|---|---|---|---|---|
| Blood | 9.1 ± 5.3 | 7.4 ± 5.5 | 4.3 ± 4.9 | 7.9 ± 1.9 | 8.9 ± 0.5 |
| Tumor | 20.9 ± 5.6 | 30.7 ± 6.6 | 48.1 ± 9.3 | 57.5 ± 5.3 | 23.5 ± 11.1 |
| Heart | 4.7 ± 2.3 | 4.5 ± 1.7 | 2.6 ± 1 | 5.9 ± 1.1 | 2.7 ± 0.2 |
| Lung | 2.3 ± 0.8 | 3.4 ± 2.9 | 2.1 ± 1.3 | 6.3 ± 0.9 | 3.7 ± 1.9 |
| Liver | 6.1 ± 2.7 | 4 ± 1.3 | 3.8 ± 1.6 | 3.2 ± 0.5 | 5.6 ± 3 |
| Spleen | 5.7 ± 1.9 | 7.5 ± 5.6 | 3.9 ± 1.4 | 4.2 ± 0.5 | 2 ± 0.3 |
| Stomach | 1.9 ± 0.3 | 1.2 ± 0.9 | 0.5 ± 0.3 | 0.8 ± 0.2 | 0.3 ± 0.1 |
| Large | 1.3 ± 0.2 | 0.8 ± 0.4 | 0.6 ± 0.4 | 0.9 ± 0.3 | 0.5 ± 0.2 |
| Intestine | | | | | |
| Small | 2.4 ± 2 | 2.7 ± 1.2 | 0.9 ± 0.1 | 1.3 ± 0.2 | 1.1 ± 0.4 |
| Intestine | | | | | |
| Kidney | 5.2 ± 0.9 | 4.9 ± 2 | 2.1 ± 1.1 | 3.3 ± 0.3 | 3 ± 0.9 |
| Muscle | 0.6 ± 0.2 | 1.7 ± 1.8 | 0.7 ± 0.3 | 3.2 ± 1 | 0.7 ± 0.3 |
| Bone | 5.4 ± 6.3 | 10.6 ± 3.5 | 9.3 ± 2.4 | 11.1 ± 5.6 | 2.6 ± 1.7 |
| Skin | 0.9 ± 0.3 | 6.9 ± 5.7 | 3.1 ± 1.6 | 7 ± 1.9 | 7.3 ± 2.1 |

Table 4 shows the tumor:tissue activity ratios for ⁸⁹Zr-DFO-DIBO/GalNAz-J591 versus time in mice bearing subcutaneous LNCaP xenografts (n=4 for each time point). Mice were administered ⁸⁹Zr-DFO-DIBO/GalNAz-J591 (0.55-0.75 MBq [15-20 Ki] in 200 μL 0.9% sterile saline) via tail vein injection (t=0). A blocking experiment was performed for the 72 h time point via co-injection of 300 μg unlabeled J591 with radiolabeled construct.

TABLE 4

| | | | | | 72 h |
|---|---|---|---|---|---|
| | 24 h | 48 h | 72 h | 96 h | block |
| Tumor: | 1.3 ± 0.6 | 3.9 ± 2.4 | 9.4 ± 2.7 | 23.3 ± 19.9 | 2.8 ± |
| Blood | | | | | 0.9 |
| Tumor: | 1 ± 0.2 | 1 ± 0.3 | 1 ± 0.1 | 1 ± 0.1 | 1 ± |
| Tumor | | | | | 0.3 |
| Tumor: | 3.2 ± 0.7 | 8.7 ± 3.8 | 18.4 ± 4.7 | 28.7 ± 9.5 | 8.9 ± |
| Heart | | | | | 2.9 |
| Tumor: | 2.7 ± 1.3 | 10.7 ± 2.8 | 10.4 ± 1.4 | 48.2 ± 8.9 | 5.2 ± |
| Lung | | | | | 1.6 |
| Tumor: | 1.4 ± 0.9 | 4.6 ± 3.6 | 11.1 ± 3.7 | 21.8 ± 6.6 | 6.1 ± |
| Liver | | | | | 1.9 |
| Tumor: | 1.3 ± 0.4 | 4 ± 3.4 | 20.7 ± 6.8 | 33.6 ± 12.3 | 8.7 ± |
| Spleen | | | | | 3.6 |
| Tumor: | 18.1 ± 5.6 | 33.3 ± 9.7 | 105.2 ± 53.7 | 91.1 ± 62.6 | 39 ± |
| Stomach | | | | | 15.1 |
| Tumor: | 14.2 ± 5.8 | 23.3 ± 24.3 | 130.9 ± 40.8 | 123.4 ± 81.2 | 57.9 ± |
| LI | | | | | 19.1 |
| Tumor: | 6 ± 2.7 | 17 ± 13.6 | 63.9 ± 16.4 | 50.1 ± 35.8 | 20.8 ± |
| SI | | | | | 10.7 |
| Tumor: | 2.9 ± 2.1 | 11.9 ± 6.7 | 21.3 ± 10.5 | 26.5 ± 12.6 | 7.3 ± |
| Kidney | | | | | 3.2 |
| Tumor: | 8.8 ± 3.1 | 12.2 ± 5.1 | 28.6 ± 14.3 | 68.2 ± 26.9 | 29.5 ± |
| Muscle | | | | | 7.9 |
| Tumor: | 1.3 ± 0.8 | 2.7 ± 0.9 | 6.5 ± 0.8 | 9.2 ± 1.3 | 8.4 ± |
| Bone | | | | | 4 |
| Tumor: | 4.7 ± 4.4 | 8.3 ± 4.4 | 18.3 ± 4.4 | 29.7 ± 7.2 | 5.7 ± |
| Skin | | | | | 1.7 |

Table 5 shows tumor:tissue activity ratios for ⁸⁹Zr-DFO-NCS-J591 versus time in mice bearing subcutaneous LNCaP xenografts (n=4 for each time point). Mice were administered ⁸⁹Zr-DFO-DIBO/GalNAz-J591 (0.55-0.75 MBq [15-20 μCi] in 200 μL 0.9% sterile saline) via tail vein injection (t=0). A blocking experiment was performed for the 72 h time point via co-injection of 300 μg unlabeled J591 with radiolabeled construct.

TABLE 5

| | 24 h | 48 h | 72 h | 96 h | 72 h block |
|---|---|---|---|---|---|
| Tumor: | 2.3 ± 1.5 | 4.2 ± 3.2 | 11.2 ± 12.8 | 7.3 ± 2.1 | 2.7 ± 1.3 |
| Blood | | | | | |
| Tumor: | 1 ± 0.4 | 1 ± 0.3 | 1 ± 0.3 | 1 ± 0.2 | 1 ± 0.7 |
| Tumor | | | | | |
| Tumor: | 4.5 ± 2.5 | 6.9 ± 3 | 18.5 ± 7.6 | 9.7 ± 2.2 | 8.6 ± 4.1 |
| Heart | | | | | |
| Tumor: | 9 ± 3.9 | 9.1 ± 7.9 | 23.1 ± 15.6 | 9.2 ± 1.9 | 6.4 ± 4.4 |
| Lung | | | | | |
| Tumor: | 3.4 ± 1.8 | 7.6 ± 2.9 | 12.7 ± 5.8 | 18 ± 3.7 | 4.2 ± 3 |
| Liver | | | | | |
| Tumor: | 3.6 ± 13.8 | 4.1 ± 3.2 | 12.5 ± 5.1 | 13.6 ± 2.5 | 12 ± 6 |
| Spleen | | | | | |
| Tumor: | 11 ± 3.3 | 24.7 ± 18.6 | 101.6 ± 60.3 | 71.2 ± 19.2 | 90.6 ± 55.6 |
| Stomach | | | | | |
| Tumor:LI | 15.9 ± 5.2 | 36.5 ± 19 | 74.2 ± 43.2 | 64.2 ± 21.1 | 51.7 ± 30.2 |
| Tumor:SI | 8.6 ± 7.4 | 11.5 ± 5.9 | 50.7 ± 11.6 | 44.8 ± 8.8 | 21.3 ± 13 |
| Tumor: | 4.1 ± 1.3 | 6.2 ± 2.9 | 22.9 ± 12.6 | 17.4 ± 3.1 | 7.8 ± 4.3 |
| Kidney | | | | | |
| Tumor: | 34.1 ± 13.2 | 17.9 ± 19.2 | 64.4 ± 27 | 38.1 ± 6.2 | 34 ± 21.9 |
| Muscle | | | | | |
| Tumor: | 3.8 ± 4.6 | 2.9 ± 1.2 | 5.2 ± 1.7 | 5.2 ± 2.7 | 9.2 ± 7.6 |
| Bone | | | | | |
| Tumor: | 22.1 ± 9.3 | 4.5 ± 3.8 | 15.4 ± 8.2 | 8.2 ± 2.5 | 3.2 ± 1.8 |
| Skin | | | | | |

Statistical Analysis:

Data were analyzed by the unpaired, two-tailed Student's t-test. Differences at the 95% confidence level (P<0.05) were considered to be statistically significant.

Discussion/Results:

For the study at hand, a model system was constructed using the anti-prostate specific membrane antigen (PSMA) antibody J591, the positron-emitting radioisotope ⁸⁹Zr (t_{1/2}=3.2 days), and its acyclic chelator desferrioxamine (DFO) (Holland et al., J. Nucl. Med. 51:1293-1300 (2010), Vugts et al., Drug Disc. Today 8:e53-e61 (2011)). This particular grouping was chosen not only because both the biology of J591 and the radiochemistry of ⁸⁹Zr are extremely well characterized but also because the system has tremendous clinical relevance, as non-site-specifically labeled ⁸⁹Zr-DFO-NCS-J591 is currently being translated to the clinic at MSKCC.

The first step in the investigation was the synthesis of the molecular components of the system. To this end, UDP-GalNAz was synthesized according to literature procedure, and DIBO-DFO was synthesized via isothiocyanate coupling of commercially available NCS-DFO and an amine-pendant DIBO (Hang et al., Proc. Natl. Acad. Sci. USA 100:14846-14851 (2003)).

With these components in hand, the antibody was then site-specifically labeled with the chelator DFO in three steps (FIG. 1). First, the antibody (1 mg) was incubated with β-1,4-galactosidase for 16 h at 37° C. in sodium phosphate buffer in order to expose the maximal number of terminal GlcNAc sugar residues. Second, the antibody was incubated with UDP-GalNAz-modified antibody (400 μg in 1 mL TBS buffer) was incubated with DIBO-DFO (200 ∥L of a 2 mM solution in DMSO) for 16 h at room temperature. After this step, purification via size exclusion chromatography yielded the final, site-specifically modified DFO-DIBO/GalNAz-J591 in 49±5% yield over three steps (n=3). As a reference for comparison, J591 was also non-site-specifically labeled with DFO via incubation of J591 with DFO-NCS (6 equivalents, Macrocyclics, Inc.) in carbonate buffer for 1 h at 37° C., followed by purification via size exclusion chromatography to obtain DFO-NCS-J591 in 86±2% yield (n=3).

SDS-PAGE experiments were run in order to assay the site-specificity of the GalNAz/DIBO-DFO conjugation methodology (FIG. 3). In these experiments, J591 that had either been left completely unmodified, modified only with GalNAz, or modified with GalNAz and subsequently clicked with DIBO-DFO were treated with PNGaseF, an amidase that cleaves at the site between the innermost GlcNAc residue and the antibody asparagines residues. As shown in the gel, after this PGNaseF treatment, the heavy chains (upper bands) of all three antibody variants were shifted to the same lower molecular weight, confirming the site-specific labeling of the heavy chain N-linked glycans.

Next, both the DFO-DIBO/GalNAz-J591 and DFO-NCS-J591 were radiolabeled with ⁸⁹Zr via incubation of antibody (400-500 μg) with ⁸⁹Zr (2.0-2.5 mCi) in PBS buffer at pH 7.0-7.5 for 1 h at room temperature, followed by purification with size exclusion chromatography, 3.4±0.3 mCi/mg for DFO-DIBO/GalNAz-J591 and DFO-NCS-J591, respectively. In further characterization, isotopic dilution experiments employing non-radioactive Zr⁴⁺ determined that the number of chelates/mAb for each variant was 2.8±0.2 for DFO-DIBO/GalNAz-J591 and 3.1±0.5 for DFO-NCS-J591. Finally, immunoreactivity experiments using the PSMA-expressing LNCaP prostate cancer cell line revealed an average immunoreactivity of 95±2% for DFO-DIBO/GalNAz-J591 and 93±2% for DFO-NCS-J591. Clearly, the properties of the site-specifically labeled J591 are identical to those of the conventionally, non-site-specifically labeled variant.

With synthesis, characterization and in vitro testing complete, the next step of the investigation was to assay the effectiveness of ⁸⁹Zr-DFO-DIBO/GalNAz-J591 in vivo. To this end, both acute biodistribution and PET imaging experiments were performed for both antibody constructs using athymic nude mice bearing subcutaneous PSMA-expressing LNCaP prostate cancer xenografts (Holland et al., J. Nucl. Med. 51:1293-1300 (2010)).

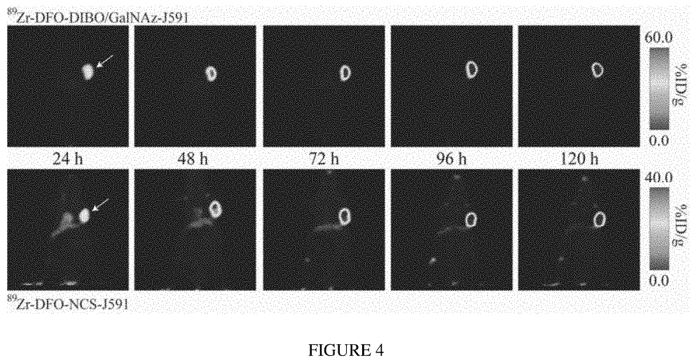

In the biodistribution experiment, nude mice bearing subcutaneous LNCaP xenografts in the shoulder were injected via tail vein with either ⁸⁹Zr-DFO-NCS-J591 or ⁸⁹Zr-DFO-DIBO/GalNAz-J591 (15-20 μCi, 4-6 μg) and were euthanized at 24, 48, 72 and 96 h post-injection, followed by the collection and weighing of tissues and assay of the amount of ⁸⁹Zr activity in each tissue (FIG. 4). For both radioimmunoconjugates, high specific uptake of the radiotracer is observed in the LNCaP xenografts, with the % ID/g increasing to a maxima at 96 h of 67.5±5.0 and 57.5±8.3 for ⁸⁹Zr-DFO-DIBO/GalNAz-J591 and ⁸⁹Zr-DFO-NCS-J591, respectively, values that yield tumor-to-muscle activity ratios at the same time point of 68.2±20.2 for ⁸⁹Zr-DFO-DIBO/GalNAz-J591 and 47.9±10.1 for ⁸⁹Zr-DFO-NCS-J591.

In terms of background uptake, the two variants behaved very similarly. As is typical of antibody-based imaging, a concomitant decrease in the % ID/g in the blood also occurred over the course of the experiment. The organs with the highest background uptake in both cases were the liver, spleen, and bone. However, by 96 h, the tumor to tissue activity ratios for each of these tissues were 21.8±6.6, 33.6±12.3, and 9.2±1.3, respectively, for ⁸⁹Zr-DFO-DIBO/GalNAz-J591, with nearly identical results for ⁸⁹Zr-DFO-NCS-J591. Importantly, blocking experiments performed by injecting a large excess (200-fold) of unlabeled J591 resulted in dramatically decreased tumor uptakes at 72 h post-injection, specifically a reduction from 56.3±5.1 to 28.5±6.8%ID/g for ⁸⁹Zr-DFO-DIBO/GalNAz-J591 and a similar drop-off for ⁸⁹Zr-DFO-NCS-J591, indicating antigen-specific in vivo targeting in both cases.

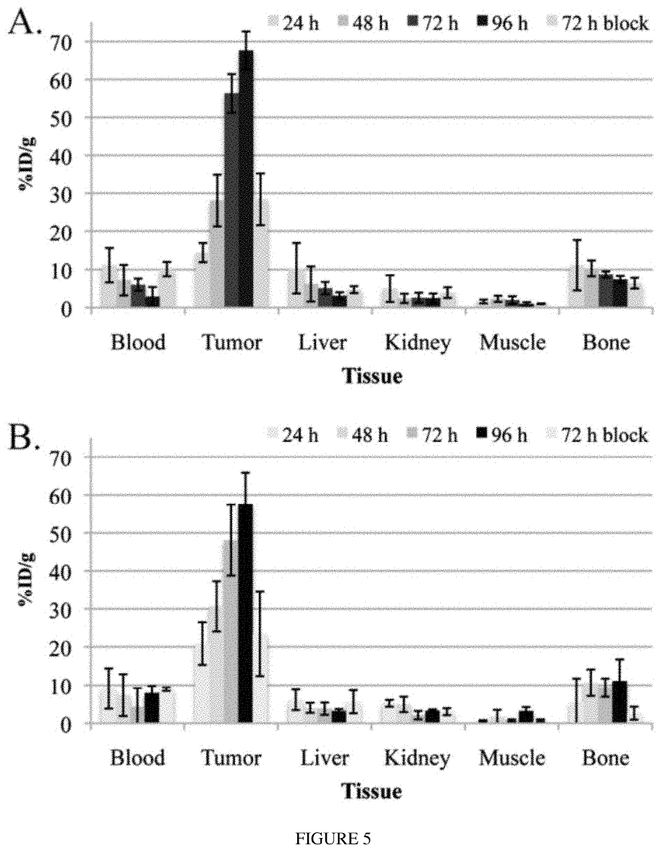

These biodistribution data were reinforced by small animal PET imaging (FIG. 5). In the imaging experiments, the results clearly indicate that the ⁸⁹Zr-DFO-DIBO/GalNAz-J591 and ⁸⁹Zr-DFO-NCS-J591 constructs are taken up significantly and selectively in the antigen-expressing LNCaP tumors. High blood pool activity and some background uptake in the heart, liver, and spleen are evident at early time points, but over the course of the experiment, the signal in the tumor increases to a point at which it is by far the most prominent feature in the image.

Plainly, theses data illustrate that the use of the site-specific conjugation methods described herein results in a final radioimmunoconjugate that is nearly identical in its in vivo behavior to its non-site-specifically labeled cousin. Indeed, both the biodistribution and small animal PET imaging results are actually suggestive of background contrast for ⁸⁹Zr-DFO-DIBO/GalNAz-J591 compared to ⁸⁹Zr-DFO-NCS-J591, without wishing to be bound by theory.

Disclosed herein are methods for the site-specific radiolabeling of antibodies on the heavy chain N-linked glycans that is predicated on both enzyme-mediated reactions and catalyst-free click chemistry. The methods disclosed herein target the heavy chain glycans as a site for specific labeling and this strategy avoids a harsh sugar oxidation step. Using the methods described herein, a “Zr-labeled radioimmunoconjugate was produced that is identical in terms of in vitro and in vivo characteristics to a similar, non-site-specifically labeled construct. Further, it is important to note that while this site-specific strategy did not result in a large improvement in immunoreactivity or in vivo behavior in this case, it may be due to the well-developed and optimized nature of the J591antibody that was used. The site-specific methods described herein will dramatically improve the in vitro and in vivo characteristics of other, less robust antibody constructs by precluding the possibility of accidental conjugation at the antigen-binding site. Although the workflow described in Example 1 involved three 16 hour incubations, the methods may be performed with two 16 hour incubations by combining the deglycosylation/glycosylation steps. Additionally, the antibody yields have been improved by optimizing the sample handling techniques. Ultimately, the methods and compositions described herein may play a critical role in the development of novel well-defined and highly specific radioimmunoconjugates in both the laboratory and the clinic.

### Example 2

### Degree of Labeling of Modified Antibodies:

Site-Specific Antibody Modification With GalNAz and Click-IT® Alexa Fluor®-488 DIBO-Alkyne.

Glycans Modification: J591 (1 mg, 8 mg/mL) underwent a buffer exchange into pre-treatment buffer (50 mM Na-phosphate, pH 6.0) using a micro-spin column prepared with P30 resin (Bio-Rad 732-6008, 1.5 mL bed volume). The column was first equilibrated in 50 mM Na-phosphate, pH 6.0, and then spun for 3 minutes at 850×g. 125 μl J591 antibody was added and then spun down for 5 minutes at 850×g. The resultant antibody solution was supplemented with 40 μL of β-1.4-galactosidase [from S. pneumonia (2 mU/μL), obtained from Life Technologies, Inc., Eugene, Oreg.] and placed in an incubator at 37° C. overnight.

GalNAz Labeling: A buffer exchange of the sample into TBS reaction buffer (20 mM Tris HCl, 0.9% NaCl, pH 7.4) was performed using a micro-spin column prepared with P30 resin. After the buffer exchange, the antibody (600 μg in 300 μL TBS buffer) was combined with UDP-GalNAz (40 μL of a 40mM solution in H₂O), MnCl₂ (150 μL of a 0.1M solution), and GalT (Y289L) (1000 μL of 0.29 mg/mL in 50 mM Tris, 5 mM EDTA (pH 8)). The final solution contained concentrations of 0.4 mg/mL antibody, 10 mM MnCl₂, 1 mM UDP-GalNAz, and 0.2 mg/mL GalT (Y289L). The resultant solution was incubated overnight at 30° C.

Click-IT® Alexa Fluor®-488 DIBO-Alkyne Ligation: The solution from the GalNAz labeling step was purified using six micro-spin columns prepared with P30 resin and TBS buffer (each micro-spin column received 250 μL of the GalNAz labeling solution). After centrifugation, the filtrates were combined to yield 1500 μL of antibody solution. Subsequently, 200 μL of Click-IT® Alexa Fluor®-488 DIBO-Alkyne solution (2 mM stock in DMSO) was added to the combined filtrates, and this tube was incubated at 25° C. overnight.

Purification: After DIBO-DFO labeling, the completed antibody was purified via size exclusion chromatography (PD10 column, GE Healthcare) and concentrated using centrifugal filter units with a 50,000 molecular weight cut off (Amicon™ Ultra 4 Centrifugal Filtration Units, Millipore Corp., Billerica, Mass.) and phosphate buffered saline (PBS, pH 7.4).

Determination of Degree of (Fluorescent) Labeling:

To determine the degree of labeling (DOL) of the fluorophore-labeled antibodies, SDS-PAGE gels were run in which the antibody being interrogated was run alongside a control antibody (GAM non-specifically labeled with Alexa Fluor® 488-SE, with a degree of labeling of 2.5 fluorophores/antibody as determined by UV-VIS spectrophotometry). For gel analysis, 200 ng of each antibody were applied on NuPAGE 4-12% Bis-Tris gels and run in MOPS buffer. Gels were imaged with FUJI FLA9000 for Alexa Fluor® 488 with an excitation of 473 nm and a 510LP filter, and then stained with SYPRO° Ruby Protein Stain and imaged with an excitation of 473 nm and a 575LP filter. The DOL of the antibody with Click-iT® DIBO-Alexa Fluor® 488 was determined using the ratio of the fluorescence intensity of Alexa Fluor® 488 to that of SYPRO® Ruby (quantitated with Multi-Gauge).

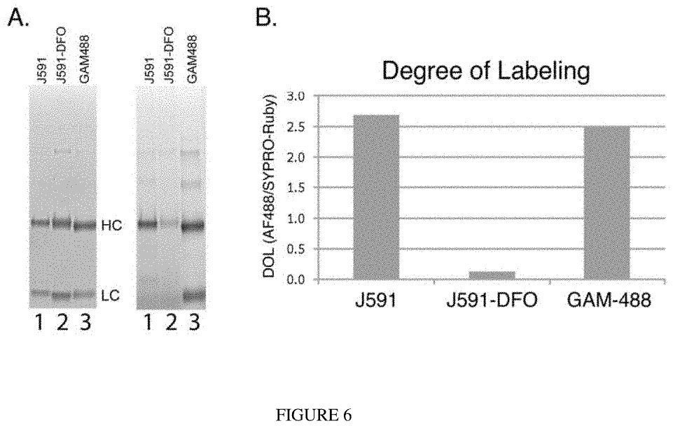

FIG. 6 shows the determination of the DOL of GalNAz-tagged J591 using a fluorescent DIBO derivative. GalNAz-modified J591 was either pre-labeled with the chelator DIBO-DFO (lane 2) or not (lane 1). As a standard, GAM non-specifically labeled with Alexa Fluor® 488-SE (DOL=2.5) was used (lane 3).

In FIG. 6, panel A, gels were imaged with FUJI FLA9000 for Alexa Fluor 488 with an excitation of 473 nm and a 510LP filter (right panel), then stained with SYPRO° Ruby Protein Stain and imaged with an excitation of 473 nm and a 575LP filter (left panel). In FIG. 6, panel B, the degree of labeling (DOL) of the antibody with Click-iT® DIBO-Alexa Fluor® 488 was determined to be 2.7±0.2 (n=3) using the ratio of the fluorescence intensity of Alexa Fluor® 488 to that of SYPRO® Ruby (quantitated with Multi-Gauge). Labeling of GalNAz-J591 with DIBO-DFO prevented >95% of dye incorporation.

Table 6 shows the reproducibility of the site-specific Gal-NAz modification of various antibodies as shown via the degree of labeling (DOL) with Click-iT® DIBO-Alexa Fluor® 488. The site-specific modification and DOL determination were performed as described above. Over the 13 different antibodies tested (n=26 individual assays), the mean DOL was 3.33±0.32.

TABLE 6

| Antibody | Isotype | Target | Degree of Labeling |
|---|---|---|---|
| Human monoclonal | IgG3 | human lymphoma cells | 3.08 |
| Human monoclonal | IgG1 | J591 | 2.70 ± 0.20 (n = 3) |
| Mouse monoclonal | IgG2a | CD4 | 3.42 ± 0.22 |
| | | | (n = 10) |
| Mouse monoclonal | IgG1 | β-tubulin | 3.38 ± 0.23 (n = 3) |
| Mouse monoclonal | IgG2a | CD3 | 2.97 |
| Mouse monoclonal | IgG2a | CD8 | 3.72 |
| Mouse monoclonal | IgG1 | CD8a | 3.71 |
| Mouse monoclonal | IgG1 | CD45 | 3.17 |
| Mouse monoclonal | IgG2a | CD56 | 2.98 |
| Mouse monoclonal | IgG1 | Complement 1 | 3.76 |
| Mouse monoclonal | IgG2a | Complement 2 | 3.60 |
| Mouse monoclonal | IgG1 | Interferon-γ | 3.41 |
| Goat polyclonal IgG | — | Apolipoprotein-A2 | 3.41 |

### Example 3

### Site-Selective Modification of a Monoclonal IgG With a DIBO PET Chelating Compound

100 μL of a 30 mg/mL stock of a monoclonal IgG is prepared in 10 mM sodium phosphate, 150 mM NaCl, pH 7.4 and deglycosylated using a deGlycIT MicroSpin column using the manufacturer's instructions (Genovis, Sweden). The deglycosylated antibody is buffer exchanged into 50 mM Tris-HCl, pH 7.4 using a 0.5 mL 50 kD MW cut-off Amicon ULTRA centrifugal filter and then diluted to 20 mg/mL in the same buffer. To a 64 μL aliquot of the antibody, 2 μL 40 mM UDP GalNAz, 1 μL 1M MnCl₂, and 8 μL 2 mg/mL GalT(Y289L) enzyme are added to a total volume of 75 μL. The solution is incubated at 30° C. for 8-16 hours. After incubation, the solution is transferred to a 0.5 mL 50 kD MW cut-off Amicon ULTRA centrifugal filter that is prewashed with Tris-buffered saline (TBS). The total volume is brought to 500 μL with TBS, and the column is centrifuged in a microfuge at 5000×g for 6 minutes. The antibody solution is brought to 500 μL with TBS and spun again at 5000×g for 6 minutes. This washing process is repeated 4 more times at which time the antibody solution is removed from the upper retentate chamber in a volume of approximately 50 μL. The antibody solution is increased to 150 μL (˜10 mg/mL) and an equal volume of 400 μM DIBO-DFO in 4% DMSO. The solution is incubated for 8-16 hours at 25° C. The antibody labeling solution is transferred to a 2.0 mL 50 kD MW cut-off Amicon ULTRA centrifugal filter that is prewashed with Tris-buffered saline (TBS) and the volume is adjusted to 2 mL with TBS and centrifuged at 1200×g for 10 minutes. The volume is adjusted to 2 mL with TBS and the sample is centrifuged at 1200×g for 10 minutes. This washing process is repeated 4 more times. The final labeled antibody solution is removed from the retentate chamber and prepared for radiolabeling experiments.

### Example 4

### Site-Selective Dual-Modal Probe Labeling of a Monoclonal IgG

A 24 mg/mL stock of a monoclonal IgG expressed from mammalian cells is prepared in 50 mM Bis-Tris, 100 mM NaCl, pH 6.0. To a 50 μL aliquot of the antibody, 10 μL of β-galactosidase (Streptococcus pneumonia, Prozyme) is added and the reaction is allowed to proceed for 4-6 hours at 37° C. After incubation, 4μL 1M Tris-HCl, pH 7.4, 2 μL 40 mM UDP GalNAz, 1 μL 1M MnCl₂, and 8 μL 2 mg/mL GalT(Y289L) enzyme are added to a total volume of 75 μL. The solution is incubated at 30° C. for 8-16 hours. After incubation, the solution is transferred to a 0.5 mL 50 kD MW cut-off Amicon ULTRA centrifugal filter that is prewashed with Tris-buffered saline (TBS). The total volume is brought to 500 μL with TBS, and the sample is centrifuged in a microfuge at 5000×g for 6 minutes. The antibody solution is increased to 500 μL with TBS and spun again at 5000×g for 6 minutes. This washing process is repeated 4 more times at which time the antibody solution is removed from the retentate chamber in a volume of approximately 50 μL. The antibody solution is increased to 150 μL (˜10 mg/mL) and an equal volume of 400 μM DIBO-DFO-AF680 dual-modal probe in 4% DMSO. The solution is incubated for 8-16 hours at 25° C. The antibody labeling solution is transferred to a 2.0 mL 50 kD MW cut-off Amicon ULTRA centrifugal filter that is prewashed with Tris-buffered saline and the volume is adjusted to 2 mL with TBS and centrifuged at 1200×g for 10 minutes. The volume is adjusted to 2 mL with TBS and the sample is centrifuged at 1200×g for 10 minutes. This washing process is repeated 4 more times. The final labeled antibody solution is removed from the retentate chamber and prepared for radiolabeling experiments.

### Example 5

### Site-Selective Dual-Modal Probe Labeling of a Monoclonal IgG

A 20 mg/mL stock of a monoclonal IgG expressed from mammalian cells is prepared in TBS and to 60 μL of the antibody solution, 4 μL 1M Tris-HCl, pH 7.4, 2 μL 40 mM UDP GalNAz, 1 μL 1M MnCl₂, and 8 μL 2 mg/mL GalT(Y289L) enzyme are added to a total volume of 75 μL. The solution is incubated at 30° C. for 8-16 hours. After incubation, the solution is transferred to a 0.5 mL 50 kD MW cut-off Amicon ULTRA centrifugal filter that is prewashed with Tris-buffered saline (TBS). The total volume is brought to 500 μL with TBS, and the sample is centrifuged in a microfuge at 5000×g for 6 minutes. The antibody solution is increased to 500 μL with TBS and spun again at 5000×g for 6 minutes. This washing process is repeated 4 more times at which time the antibody solution is removed from the retentate chamber in a volume of approximately 50 μL. The antibody solution is increased to 150 μL (˜10 mg/mL) and an equal volume of 400 μM DIBO-AF680 fluorescent probe in 4% DMSO, and the solution is incubated for 8-16 hours at 25° C. The antibody labeling solution is transferred to a 2.0 mL 50 kD MW cut-off Amicon ULTRA centrifugal filter that is prewashed with 50 mM Bis-Tris, 100 mM NaCl, pH 6.0 and the volume is adjusted to 2 mL with 50 mM Bis-Tris, 100 mM NaCl, pH 6.0 and centrifuged at 1200×g for 10 minutes. The volume is adjusted to 2 mL with the same buffer and the sample is centrifuged at 1200×g for 10 minutes. This washing process is repeated 3 more times and the sample is spun down to a final volume of 50 μL. The sample is removed to a microcentrifuge tube and 10 μL of β-galactosidase is added and the reaction is allowed to proceed for 4-6 hours at 37° C. After incubation, 4 μL 1M Tris-HCl, pH 7.4, 2 μL 40 mM UDP GalNAz, 1 μL 1M MnCl₂, and 8 μL 2 mg/mL GalT(Y289L) enzyme are added to a total volume of 75 μL. The solution is incubated at 30° C. for 8-16 hours. After incubation, the solution is transferred to a 0.5 mL 50 kD MW cut-off Amicon ULTRA centrifugal filter that is prewashed with Tris-buffered saline (TBS). The total volume is brought to 500 μL with TBS, and the sample is centrifuged in a microfuge at 5000×g for 6 minutes. The antibody solution is increased to 500 μL with TBS and spun again at 5000×g for 6 minutes. This washing process is repeated 4 more times at which time the antibody solution is removed from the retentate chamber in a volume of approximately 50 μL. The antibody solution is increased to 150 μL (˜10 mg/mL) and an equal volume of 400 μM DIBO-DFO probe in 4% DMSO. The solution is incubated for 8-16 hours at 25° C. The antibody labeling solution is transferred to a 2.0 mL 50 kD MW cut-off Amicon ULTRA centrifugal filter that is prewashed with Tris-buffered saline and the volume is adjusted to 2 mL with TBS and centrifuged at 1200×g for 10 minutes. This washing process is repeated 4 more times. The final labeled antibody solution is removed from the retentate chamber and prepared for radiolabeling experiments.

### Example 6

### Site-Selective Dual-Modal Probe Labeling of a Monoclonal IgG

A 24 mg/mL stock of a monoclonal IgG expressed from mammalian cells is prepared in 50 mM Bis-Tris, 100 mM NaCl, pH 6.0. To a 50 μL aliquot of the antibody, 10 μL of β-galactosidase and the reaction is allowed to proceed for 4-6 hours at 37° C. After incubation, 4 μL 1M Tris-HCl, pH 7.6, 2μL 40 mM UDP GalNAz, 1 μL 1M MnCl₂, and 8 μL 2 mg/mL GalT(Y289L) enzyme are added to a total volume of 75 μL. The solution is incubated at 30° C. for 8-16 hours. After incubation, the solution is transferred to a 0.5 mL 50 kD MW cut-off Amicon ULTRA centrifugal filter that is prewashed with Tris-buffered saline (TBS). The total volume is brought to 500 μL with TBS, and the sample is centrifuged in a microfuge at 5000×g for 6 minutes. The antibody solution is increased to 500 μL with TBS and spun again at 5000×g for 6 minutes. This washing process is repeated 4 more times at which time the antibody solution is removed from the retentate chamber in a volume of approximately 50 μL. The antibody solution is increased to 150 mg/mL) and an equal volume of a solution containing 100-400 μM DIBO-AF680 and 400-100 μM DIBO-DFO probe in 4% DMSO. The solution is incubated for 8-16 hours at 25° C. The antibody labeling solution is transferred to a 2.0 mL 50 kD MW cut-off Amicon ULTRA centrifugal filter that is prewashed with Tris-buffered saline and the volume is adjusted to 2 mL with TBS and centrifuged at 1200×g for 10 minutes. This washing process is repeated 4 more times. The final labeled antibody solution is removed from the upper retentate chamber and prepared for radiolabeling experiments.

1-104. (canceled)

105. A labeling molecule having the formula:
FLUOROPHORE-REACTIVE GROUP-METAL ION CHELATOR.

106. The labeling molecule of claim 105, wherein
FLUOROPHORE is a coumarin, a cyanine, a benzofuran, a quinolone, a quinazoline, an indole, a benzazole, a borapolyazaindacine, or a xanthene;
REACTIVE GROUP comprises a terminal triarylphosphine, an alkyne, a terminal alkyne, an activated alkyne group, an azide, a ketone, a hydrazide, a semicarbazide, a thiocarbonylhydrazide, a carbonylhydrazide, a thiocarbonylhydrazide, a sulfonylhydrazide, a carbazide, a thiocarbazide, an aminooxy group, a Diels-Alder diene, or a Diels-Alder dienophile; and
METAL ION CHELATOR is 1,4,8,11-tetraazabicyclo[6.6.2]hexadecane-4,11-diyl)diacetic acid (CB-TE2A), desferrioxamine (DFO), diethylenetriaminepentaacetic acid (DTPA), 1,4,7,10-tetraazacyclotetradecane-1,4,7,10-tetraacetic acid (DOTA), ethylenediaminetetraacetic acid (EDTA), ethylene glycolbis(2-aminoethyl)-N,N,N′,N′-tetraacetic acid (EGTA), 1,4,8,11-tetraazacyclotetradecane-1,4,8,11-tetraacetic acid (TETA), ethylenebis-(2-4 hydroxy-phenylglycine) (EHPG), 5-Cl-EHPG, 5Br-EHPG, 5-Me-EHPG, 5t-Bu-EHPG, 5-sec-Bu-EHPG, benzodiethylenetriamine pentaacetic acid (benzo-DTPA), dibenzo-DTPA, phenyl-DTPA, diphenyl-DTPA, benzyl-DTPA, dibenzyl DTPA, bis-2 (hydroxybenzyl)-ethylene-diaminediacetic acid (HBED) and derivatives thereof, Ac-DOTA, benzo-DOTA, dibenzo-DOTA, 1,4,7-triazacyclononane N,N′,N”-triacetic acid (NOTA), benzo-NOTA, benzo-TETA, benzo-DOTMA, where DOTMA is 1,4,7,10-tetraazacyclotetradecane-1,4,7,10-tetra(methyl tetraacetic acid), benzo-TETMA, where TETMA is 1,4,8,11-tetraazacyclotetradecane-1,4,8,11-(methyl tetraacetic acid), derivatives of 1,3-propylenediaminetetraacetic acid (PDTA), triethylenetetraaminehexaacetic acid (TTHA), derivatives of 1,5,10-N,N,N″-tris(2,3-dihydroxybenzoyl)-tricatecholate (LICAM), or 1,3,5-N,N′,N″-tris(2,3-dihydroxybenzoyl)aminomethylbenzene (MECAM).

107. The labeling molecule of claim 105, wherein the labeling molecule comprises a tyrosine moiety.

108. The labeling molecule of claim 105, wherein the REACTIVE GROUP comprises an activated alkyne group.

109. The labeling molecule of claim 108, wherein the activated alkyne group is 4-dibenzocyclooctynol (DIBO).

110. The labeling molecule of claim 105, wherein the METAL ION CHELATOR is desferrioxamine (DFO), 1,4,7-triazacyclononane N,N,N″-triacetic acid (NOTA) or 1,4,7,10-tetraazacyclotetradecane-1,4,7,10-tetraacetic acid (DOTA).

111. The labeling molecule of claim 105, wherein the FLUOROPHORE is a xanthene, a coumarine, a borapolyazaindacine, or a cyanine and the REACTIVE GROUP comprises an activated alkyne group, and the METAL ION CHELATOR is desferrioxamine (DFO), 1,4,7-triazacyclononane N,N′,N″-triacetic acid (NOTA) or 1,4,7,10-tetraazacyclotetradecane-1,4,7,10-tetraacetic acid (DOTA).

112. The labeling molecule of claim 111, wherein the activated alkyne group is a cyclooctyne.

113. The labeling molecule of claim 112, wherein the cyclooctyne is 4-dibenzocyclooctynol (DIBO).

114. A kit comprising:
a modified sugar comprising a chemical handle;
a labeling molecule comprising a metal ion chelator group, a reactive group, and a fluorophore; and
instructions for using.

115. A method for labeling a glycoprotein, the method comprising:
a) providing a glycoprotein comprising a terminal GlcNAc residue;
b) providing a modified sugar comprising a chemical handle;
c) contacting the glycoprotein with the modified sugar, wherein the modified sugar attaches to the terminal GlcNAc residue to provide a modified glycoprotein;
d) providing a labeling molecule having a formula FLUOROPHORE-REACTIVE GROUP-METAL ION CHELATOR;
e) contacting the modified glycoprotein with the labeling molecule, wherein the reactive group attaches to the chemical handle to provide a labeled glycoprotein;
f) providing a radioactive metal ion; and
g) contacting the labeled glycoprotein with the radioactive metal ion, wherein the metal ion associates with the chelator group to provide a radiolabeled glycoprotein.
